(19) United States
(12) Patent Application Publication
(10) Pub. No.: US 2018/0208928 A1
(43) Pub. Date: Jul. 26, 2018

(54) TOOLS AND METHODS USING MIRNA 182, 96 AND/OR 183 FOR TREATING PATHOLOGIES

(71) Applicants: Volker Busskamp, Newton, MA (US); Witold Filipowicz, Riehen (CH); Jacek Krol, Basel (CH); Botond Roska, Oberwil (CH)

(72) Inventors: Volker Busskamp, Newton, MA (US); Witold Filipowicz, Riehen (CH); Jacek Krol, Basel (CH); Botond Roska, Oberwil (CH)

(73) Assignee: FRIEDRICH MIESCHER INSTITUTE FOR BIOMEDICAL RESEARCH, BASEL (CH)

(21) Appl. No.: 15/833,843

(22) Filed: Dec. 6, 2017

Related U.S. Application Data
(62) Division of application No. 15/024,289, filed on Mar. 23, 2016, now abandoned.

(30) Foreign Application Priority Data
Sep. 26, 2013 (EP) 13186141.1

(51) Int. Cl. C12N 15/113

(52) U.S. Cl. CPC C12N 15/113; C12N 2330/10; C12N 2310/141

(57) ABSTRACT

The present inventions relates to isolated nucleic acid molecules comprising a nucleotide sequence coding for miRNA-182 (uuuggcaaugguagaacucacacu or ugguucuagacuugccaacua), miRNA-96 (uuuggcacuagcacauuuuugcu or aaucaugugcagugccaauaug) and/or miRNA-183 (uauggcacugguagaauucacu or gugaauuaccgaagggccauaa) for use in treating or ameliorating a ciliopathy and/or a photoreceptor dysfunction.

### FIELD OF THE INVENTION

The present invention relates to methods of treating pathologies such as ciliopathies or photoreceptor degenerations. The present invention also relates to agents for use treating pathologies such as ciliopathies or photoreceptor degenerations, as well as their use in the manufacture of a medicament.

### BACKGROUND OF THE INVENTION

Blindness is a major health problem that disables millions of people worldwide. The most common cause of blindness is the disfunction of the retina. The three most common forms of retinal blindness are retinitis pigmentosa (RP), macular degeneration (MD) and glaucoma (G).

In RP and MD the primary problem is the degeneration of photoreceptors and the consequent loss of photosensitivity. There is thus a need to be able to obviate the problems associated with such degeneration of photoreceptors.

Cones, which are the photoreceptors for daylight visual activity, capture light using their outer segment, an organelle derived from primary cilia (J. N. Pearring, R. Y. Salinas, S. A. Baker, V. Y. Arshavsky, Protein sorting, targeting and trafficking in photoreceptor cells, Prog Retin Eye Res 36, 24-51 (2013)). In adults, the loss of cone outer segments or their function is the final common pathway in most photoreceptor diseases which cause blindness (J.-A. Sahel, B. Roska, Gene therapy for blindness, Annu. Rev. Neurosci. 36, 467-488 (2013); T. Léveillard, J.-A. Sahel, Rod-derived cone viability factor for treating blinding diseases: from clinic to redox signaling, Sci Transl Med 2, 26ps16 (2010)). To preserve these organelles, or to regenerate them, requires knowledge of the molecular pathways that control their maintenance in normal adults (T. Léveillard, J.-A. Sahel, Rod-derived cone viability factor for treating blinding diseases: from clinic to redox signaling, Sci Transl Med 2, 26ps16 (2010)). MicroRNAs (miRNAs) are posttranscriptional repressors of gene expression. Their biogenesis occurs in two steps. The primary RNA transcripts pri-miRNAs are cleaved by the DROSHA/DGCR8 complex into pre-miRNAs, which are further processed by DICER to become mature miRNAs (J. Krol, I. Loedige, W. Filipowicz, The widespread regulation of microRNA biogenesis, function and decay, Nat. Rev. Genet. 11, 597-610 (2010)). During retinal development the lack of all or particular miRNAs lead to various defects (T. R. Sundermeier, K. Palczewski, The physiological impact of microRNA gene regulation in the retina, Cellular and Molecular Life Sciences 69, 2739-2750 (2012)) including retinal degeneration (D. Damiani et al., Dicer inactivation leads to progressive functional and structural degeneration of the mouse retina, J. Neurosci. 28, 4878-4887 (2008); Q. Zhu et al., Sponge Transgenic Mouse Model Reveals Important Roles for the MicroRNA-183 (miR-183)/96/182 Cluster in Postmitotic Photoreceptors of the Retina, J. Biol. Chem. 286, 31749-31760 (2011); S. Lumayag et al., Inactivation of the microRNA-183/96/182 cluster results in syndromic retinal degeneration, Proc. Natl. Acad. Sci. U.S.A. 110, E507-516 (2013)) or cone death (R. Sanuki et al., miR-124a is required for hippocampal axogenesis and retinal cone survival through Lhx2 suppression, Nat. Neurosci. 14, 1125-1134 (2011)). In adult photoreceptors, few miRNAs are highly expressed (M. Karali et al., miRNeye: a microRNA expression atlas of the mouse eye, BMC Genomics 11, 715 (2010)), and some are regulated by light (J. Krol et al., Characterizing light-regulated retinal microRNAs reveals rapid turnover as a common property of neuronal microRNAs, Cell 141, 618-631 (2010)).

### SUMMARY OF THE INVENTION

In order to investigate the contribution of miRNAs to the function of adult cones, the present inventors depleted all miRNAs from developed and functional cones in vivo. They observed that the cone-specific disruption of DGCR8 in adult mice led to the loss of miRNAs, the downregulation of cone opsins, and the loss of outer segments resulting in photoreceptors that were unable to detect light. They also observed that, surprisingly, the number of cones remained unchanged but that they lost their genetic signature. Even more surprising, they found that the mere re-expression of the sensory-cell-specific miR-182 and 183 not only prevented outer segment loss, but that these miRNAs were sufficient to induce the formation of outer segments in stem cell-derived retinal cultures, which are normally deprived of such outer segment.

The present invention hence encompasses an isolated nucleic acid molecule comprising a nucleotide sequence coding for miRNA-182 (uuuggcaaugguagaacucacacu, SEQ ID NO:1, or ugguucuagacuugccaacua, SEQ ID NO:2), miRNA-96 (uuuggcacuagcacauuuuugcu, SEQ ID NO:5, or aaucaugugcagugccaauaug, SEQ ID NO:6) and/or miRNA-183 (uauggcacugguagaauucacu, SEQ ID NO:3, or gugaauuaccgaagggccauaa, SEQ ID NO:4) for use, e.g. as a medicament, in treating or ameliorating a ciliopathy. In some embodiments, the ciliopathy will be selected from the group comprising Senior-Løken syndrome, retinal degeneration, retinitis pigmentosa.

Given that loss of outer segments can also be due to diseases and degenerations which are not typically known as ciliopathy, the present invention also encompasses an isolated nucleic acid molecule comprising a nucleotide sequence coding for miRNA-182 (uuuggcaaugguagaacucacacu, SEQ ID NO:1, or ugguucuagacuugccaacua, SEQ ID NO:2), miRNA-96 (uuuggcacuagcacauuuuugcu, SEQ ID NO:5, or aaucaugugcagugccaauaug, SEQ ID NO:6) and/or miRNA-183 (uauggcacugguagaauucacu, SEQ ID NO:3, or gugaauuaccgaagggccauaa, SEQ ID NO:4) for use, as e.g. a medicament, in treating or ameliorating a photoreceptor dysfunction. In some embodiments, said photoreceptor dysfunction will be selected from the group comprising comprising achromatic vision, macular degeneration, retinitis pigmentosa, rod and/or cone dystrophy, and Usher syndrome.

In some embodiments, the isolated nucleic acid molecule is miRNA 182. In some it is miRNA 183. In other embodiments, it is miRNA 96. In other embodiments, it is a combination of all three is miRNA 182, miRNA 183 and miRNA 96. In yet other embodiments, it is a combination of miRNA 182 and miRNA 183. In other embodiments, it is a combination of miRNA 182 and miRNA 96. In yet other embodiments, it is a combination of miRNA 183 and miRNA 96

The isolated nucleic acid molecule can be in any vehicle suitable for administration to a subject, e.g. a liposome, a polymer microsphere, a liposome, a colloidal gold particle, a lipopolysaccharide, a polypeptide, a polysaccharide, a pegylated virus vehicle, a nanoparticle or any kind of delivery vehicle. The isolated nucleic acid molecule can also be included recombinant vector able to deliver it to cells of the subject to be treated. The present invention also encompasses a host cell such a recombinant vector.

The present invention further encompasses a therapeutic composition for increasing expression of miRNA-182, miRNA-96 and/or miRNA-183 in a cell, wherein the composition comprises a nucleic acid or a vector according to the invention, said nucleic acid or vector providing for expression of the miRNA in the cell. Typically, such a composition will further comprise a pharmaceutically acceptable carrier, diluent, or buffer. The delivery vehicles mentioned hereinabove are also suitable for the delivery of such therapeutic composition.

The present invention also encompasses a method for treating a disease or condition associated with the downregulation of miRNA-182, miRNA-96 and/or miRNA-183, such as ciliopathy or photoreceptor dysfunction as described hereinabove, in a subject, the method comprising administering to the subject an effective amount of an agent according to the invention that increases expression of miRNA-182, miRNA-96 and/or miRNA-183 in the subject. As evident for the skilled person, such methods of treatment might include administering to the subject additional factors such as Argonaute, Rod-derived Cone Viability Factor (RdCVF), and/or a nucleic acid molecule coding for such an additional factor.

### DESCRIPTION OF THE FIGURES

FIG. 1: Rescue of opsin expression in C-DGCR-KO cones. Confocal side projections of OPN1SW/MW merged with AAV-transfected cones (left panel), merged with tdTomato-labeled cones (middle panel) and all channels merged (right panel). Sh-Control (upper row), sh-miR-124 (middle row) and sh-miR-183/182 (bottom row) are shown. The outer segments (OS) are indicated by the dashed line. (G) Topviews on OPN1SW/MW-positive outer segments (green) of sh-Control (left panel), sh-miR-124 (middle panel) and sh-miR-183/182 (right panel). (H) Quantification of OPN1SW/MW foci shown in (G). (I) Embryonic stem (ES) cell-derived retina section at day 27 in culture. Photoreceptors are labeled with recoverin (magenta), bipolar cells with Chx10 (green) antibodies. Cell nuclei are labeled with Hoechst (white). (J-K) These two figures show the upper part of the photoreceptor layer of ES cell-derived retinas. (J) ES cell-derived retina culture at day 27 labeled with anti-rhodopsin antibody (red) and Hoechst (white) infected with AAV expressing control RNA at day 7. (K) ES cell-derived retina culture was infected with AAV expressing pri-miR-183/96/182 at day 7 and then labeled with anti-rhodopsin antibody (red) and Hoechst (white) at day 27.

FIG. 2: Rescue of opsin expression in C-DGCR-KO cones. Topviews on OPN1SW/MW-positive outer segments (green) of sh-Control (left panel), sh-miR-124 (middle panel) and sh-miR-183/182 (right panel).

FIG. 3: Rescue of opsin expression in C-DGCR-KO cones. (H) Quantification of OPN1SW/MW foci shown in FIG. 2.

FIG. 4: A: Embryonic stem (ES) cell-derived retina section at day 27 in culture. Photoreceptors are labeled with recoverin, bipolar cells with Chx10 antibodies. Cell nuclei are labeled with Hoechst. B, C: These two panels show the upper part of the photoreceptor layer of ES cell-derived retinas. B: ES cell-derived retina culture at day 27 labeled with anti-rhodopsin antibody and Hoechst infected with AAV expressing control RNA at day 7. C: ES cell-derived retina culture was infected with AAV expressing pri-miR-183/96/182 at day 7 and then labeled with anti-rhodopsin antibody and Hoechst at day 27.

FIG. 5: Opsin expression in the rd1 mouse model is rescued by microRNA cluster 183/96/182. A: Topview of OPN1SW/MW-positive outer segments (red) in control uninjected retina of the rd1 mouse (P38). B: Topview of OPN1SW/MW-positive outer segments (red) showing rescue of opsin expression by scAAV encoding microRNA cluster-DsRed2. DsRed2 (magenta) labels the infected cells (P38).

FIG. 6: MiR-183/96/182 cluster induces the formation of short outer segments as well as light responses in ES cells-derived retinal cultures. (A) Representative EM images of distal structures of the d25 ES cells-derived retina culture that was infected at d7 with AAV expressing pri-miR-183/96/182. Upper picture shows an enlarged EM image of a short outer segment including disk membranes. A longitudinal section of an entire inner segment, connecting cilium and outer segment (bottom left) and a cross section of the connecting cilium (bottom right) are also shown, with asterisks highlighting the nine microtubule bundles. Scale bars as indicated. (B) Infrared image of a slice of an ES cells-derived retinal culture from which we performed electrophysiological recordings. (C) An example of a hyperpolarizing response from a recorded photoreceptor in response to full-field light stimulation. The gray bar indicates the timing of stimulation (top panel). Quantification of peak responses (bottom panel).

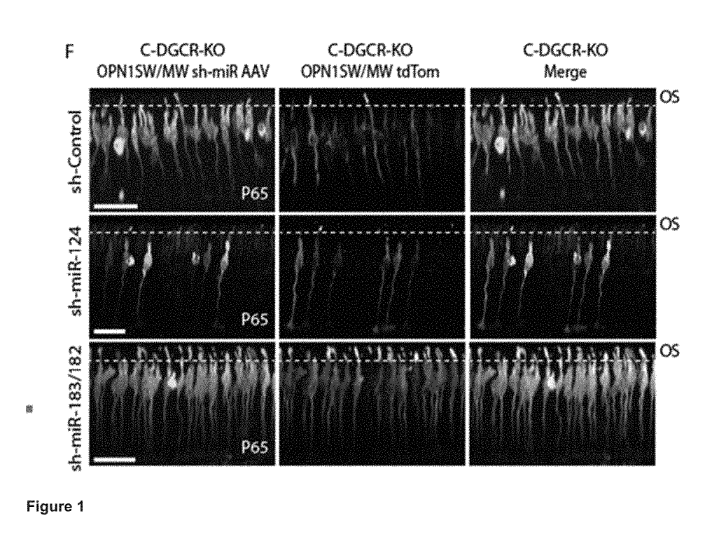

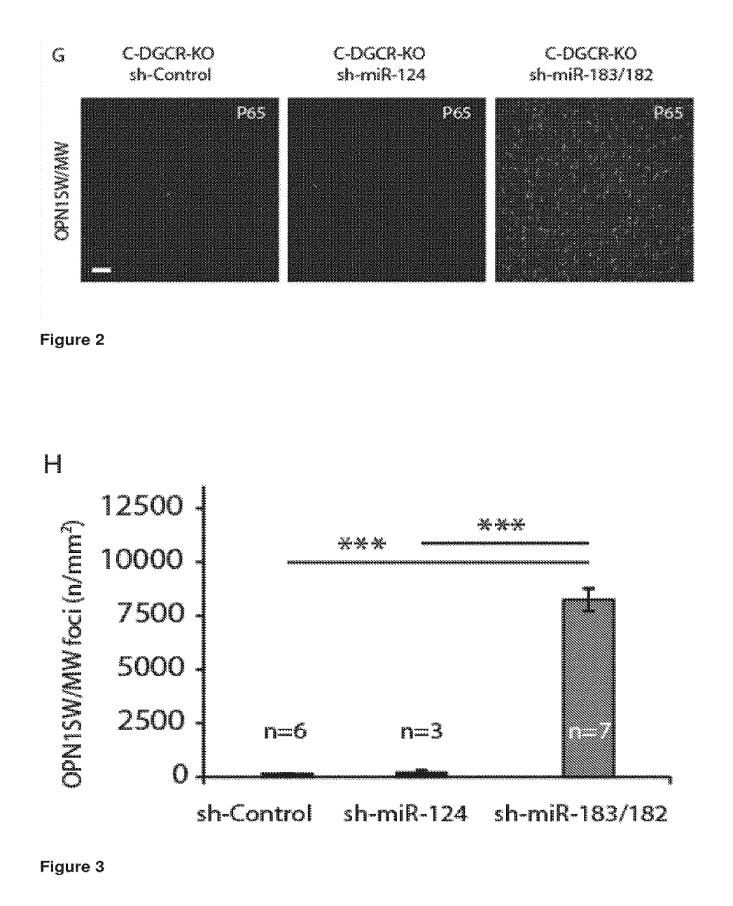

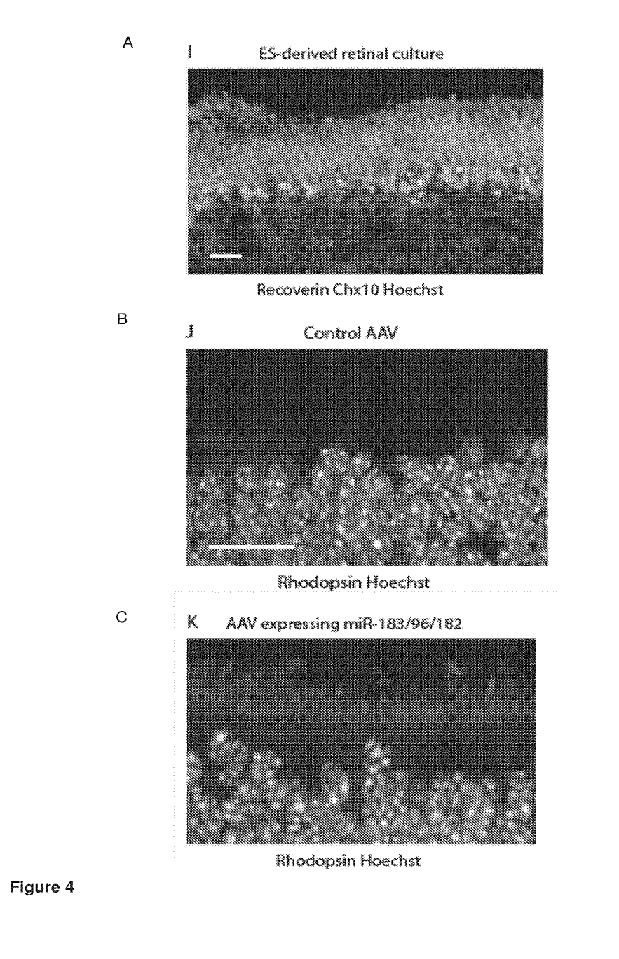

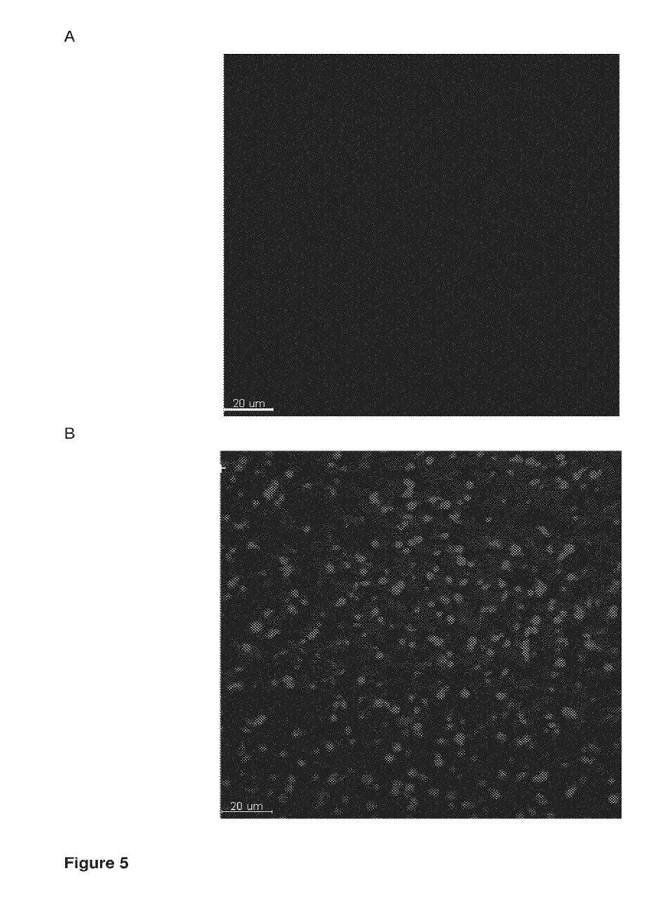

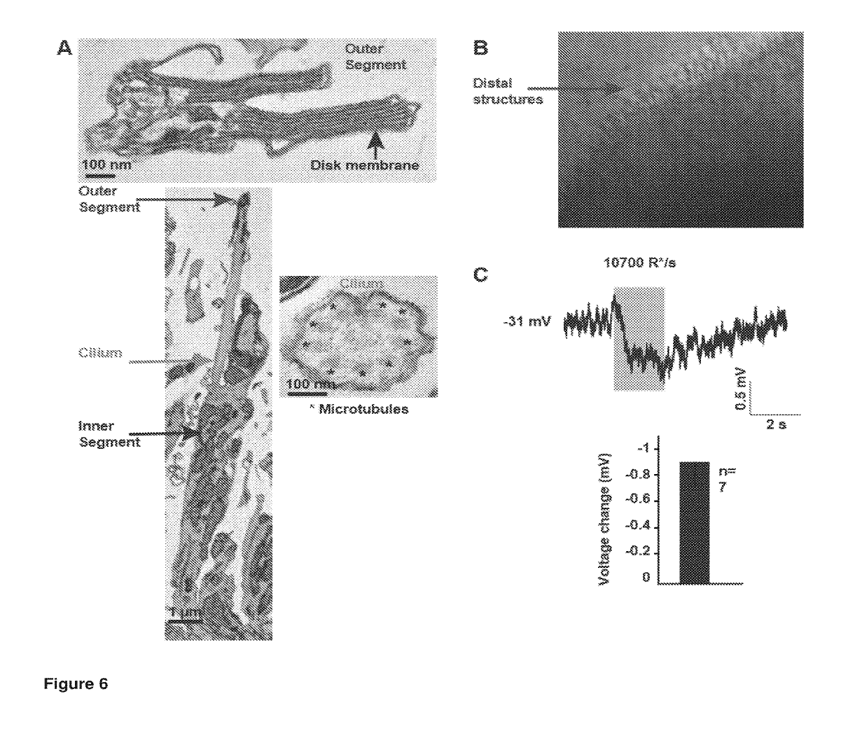

### DETAILED DESCRIPTION OF THE INVENTION

In order to investigate the contribution of miRNAs to the function of adult cones, the present inventors depleted all miRNAs from developed and functional cones in vivo. They observed that the cone-specific disruption of DGCR8 in adult mice led to the loss of miRNAs, the downregulation of cone opsins, and the loss of outer segments resulting in photoreceptors that were unable to detect light. They also observed that, surprisingly, the number of cones remained unchanged but that they lost their genetic signature. Even more surprising, they found that the mere re-expression of the sensory-cell-specific miR-182 and 183 not only prevented outer segment loss, but that these miRNAs were sufficient to induce the formation of outer segments in stem cell-derived retinal cultures, which are normally deprived of such outer segment. The present invention hence encompasses an isolated nucleic acid molecule comprising a nucleotide sequence coding for miRNA-182 (uuuggcaaugguagaacucacacu, SEQ ID NO:1, or ugguucuagacuugccaacua, SEQ ID NO:2), miRNA-96 (uuuggcacuagcacauuuuugcu, SEQ ID NO:5, or aaucaugugcagugccaauaug, SEQ ID NO:6) and/or miRNA-183 (uauggcacugguagaauucacu, SEQ ID NO:3, or gugaauuaccgaagggccauaa, SEQ ID NO:4) for use, e.g. as a medicament, in treating or ameliorating a ciliopathy. In some embodiments, the ciliopathy will be selected from the group comprising Senior-Løken syndrome, retinal degeneration, retinitis pigmentosa.

Given that loss of outer segments can also be due to diseases and degenerations which are not typically known as ciliopathy, the present invention also encompasses an isolated nucleic acid molecule comprising a nucleotide sequence coding for miRNA-182 (uuuggcaaugguagaacucacacu, SEQ ID NO:1, or ugguucuagacuugccaacua, SEQ ID NO:2), miRNA-96 (uuuggcacuagcacauuuuugcu, SEQ ID NO:5, or aaucaugugcagugccaauaug, SEQ ID NO:6) and/or miRNA-183 (uauggcacugguagaauucacu, SEQ ID NO:3, or gugaauuaccgaagggccauaa, SEQ ID NO:4) for use, as e.g. a medicament, in treating or ameliorating a photoreceptor dysfunction. In some embodiments, said photoreceptor dysfunction will be selected from the group comprising comprising achromatic vision, macular degeneration, retinitis pigmentosa, rod and/or cone dystrophy, and Usher syndrome.

In some embodiments, the isolated nucleic acid molecule is miRNA 182. In some it is miRNA 183. In other embodiments, it is miRNA 96. In other embodiments, it is a combination of all three is miRNA 182, miRNA 183 and miRNA 96. In yet other embodiments, it is a combination of miRNA 182 and miRNA 183. In other embodiments, it is a combination of miRNA 182 and miRNA 96. In yet other embodiments, it is a combination of miRNA 183 and miRNA 96 The isolated nucleic acid molecule can be in any vehicle suitable for administration to a subject, e.g. a liposome, a polymer microsphere, a liposome, a colloidal gold particle, a lipopolysaccharide, a polypeptide, a polysaccharide, a pegylated virus vehicle, a nanoparticle or any kind of delivery vehicle. The isolated nucleic acid molecule can also be included recombinant vector able to deliver it to cells of the subject to be treated. The present invention also encompasses a host cell such a recombinant vector.

The present invention further encompasses a therapeutic composition for increasing expression of miRNA-182, miRNA-96 and/or miRNA-183 in a cell, wherein the composition comprises a nucleic acid or a vector according to the invention, said nucleic acid or vector providing for expression of the miRNA in the cell. Typically, such a composition will further comprise a pharmaceutically acceptable carrier, diluent, or buffer. The delivery vehicles mentioned hereinabove are also suitable for the delivery of such therapeutic composition.

The present invention also encompasses a method for treating a disease or condition associated with the downregulation of miRNA-182, miRNA-96 and/or miRNA-183, such as ciliopathy or photoreceptor dysfunction as described hereinabove, in a subject, the method comprising administering to the subject an effective amount of an agent according to the invention that increases expression of miRNA-182, miRNA-96 and/or miRNA-183 in the subject. As evident for the skilled person, such methods of treatment might include administering to the subject additional factors such as Argonaute, Rod-derived Cone Viability Factor (RdCVF), and/or a nucleic acid molecule coding for such an additional factor.

These and other aspects of the present invention should be apparent to those skilled in the art, from the teachings herein.

It is to be understood that a medicament in the sense of the present invention is generally used therapeutically, but it may be used in a prophylactic sense, when a subject has been identified as being likely to suffer from blindness, but actual vision loss has not yet occurred or has only minimally occurred.

By “blindness” is meant total or partial loss of vision. Typically the medicament may be used to treat blindness associated with macular degeneration, glaucoma and/or retinitis pigmentosa. However, it is to be appreciated that any disease or condition which leads to degeneration or loss of the outer segments of photoreceptors in the eye may be treated using the medicament. Moreover, without wishing to be bound by theory, it is believed that the present invention will be particularly effective for curing blindness at early stages of retinal degeneration (rd) when photoreceptor function is lost but the photoreceptor-to-bipolar synapse may still be intact.

It will be appreciated that the present invention also extends to methods of treating prophylactically or therapeutically blindness by administering to a patient suffering or predisposed to developing blindness, a DNA construct according to the invention.

Typically, the medicament according to the present invention may be administered to a subject in the form of a recombinant molecule comprising the miRNA to allow expression of said miRNA in the photoreceptors of the subject. It will be appreciated that the nucleic acid coding for the miRNA of the invention may be under control of a suitable promoter, such as a constitutive and/or controllable promoter.

Many different viral and non-viral vectors and methods of their delivery, for use in gene therapy, are known, such as adenovirus vectors, adeno-associated virus vectors, retrovirus vectors, lentiviral vectors, herpes virus vectors, liposomes, naked DNA administration and the like. A detailed review of possible techniques for transforming genes into desired cells of the eye is taught by Wright (Br J Ophthalmol, 1997; 81: 620-622) which is incorporated herein by reference. Moreover, it may also be possible to use encapsulated cell technology as developed by Neurotech, for example.

Preferably the cells to which the medicament or vector are to be administered, and in which the gene is to be expressed are photoreceptors, i.e. rods or cones. Moreover, the photoreceptor cells (rod and cones) which have lost photosensitivity due to degradation of their outer segment, but which are not “dead” can be used to express the miRNA of the invention. Moreover expression of the miRNA of the invention in photoreceptors may serve to prevent or show down degeneration.

For convenience, the meaning of certain terms and phrases employed in the specification, examples, and appended claims are also provided below.

The singular forms “a,” “an,” and “the” include plural reference unless the context clearly dictates otherwise.

“Polynucleotide” and “nucleic acid”, used interchangeably herein, refer to polymeric forms of nucleotides of any length, either ribonucleotides or deoxyribonucleotides. Thus, these terms include, but are not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases. These terms further include, but are not limited to, mRNA or cDNA that comprise intronic sequences. The backbone of the polynucleotide can comprise sugars and phosphate groups (as may typically be found in RNA or DNA), or modified or substituted sugar or phosphate groups. Alternatively, the backbone of the polynucleotide can comprise a polymer of synthetic subunits such as phosphoramidites and thus can be an oligodeoxynucleoside phosphoramidate or a mixed phosphoramidate-phosphodiester oligomer. A polynucleotide may comprise modified nucleotides, such as methylated nucleotides and nucleotide analogs, uracyl, other sugars, and linking groups such as fluororibose and thioate, and nucleotide branches. The sequence of nucleotides may be interrupted by non-nucleotide components. A polynucleotide may be further modified after polymerization, such as by conjugation with a labeling component. Other types of modifications included in this definition are caps, substitution of one or more of the naturally occurring nucleotides with an analog, and introduction of means for attaching the polynucleotide to proteins, metal ions, labeling components, other polynucleotides, or a solid support. The term “polynucleotide” also encompasses peptidic nucleic acids, PNA and LNA. Polynucleotides may further comprise genomic DNA, cDNA, or DNA-RNA hybrids.

“Sequence Identity” refers to a degree of similarity or complementarity. There may be partial identity or complete identity. A partially complementary sequence is one that at least partially inhibits an identical sequence from hybridizing to a target polynucleotide; it is referred to using the functional term “substantially identical”. The inhibition of hybridization of the completely complementary sequence to the target sequence may be examined using a hybridization assay (Southern or Northern blot, solution hybridization and the like) under conditions of low stringency. A substantially identical sequence or probe will compete for and inhibit the binding (i.e., the hybridization) of a completely identical sequence or probe to the target sequence under conditions of low stringency. This is not to say that conditions of low stringency are such that non-specific binding is permitted; low stringency conditions require that the binding of two sequences to one another be a specific (i.e., selective) interaction. The absence of non-specific binding may be tested by the use of a second target sequence which lacks even a partial degree of complementarities (e.g., less than about 30% identity); in the absence of non-specific binding, the probe will not hybridize to the second non-complementary target sequence.

Another way of viewing sequence identity in the context to two nucleic acid or polypeptide sequences includes reference to residues in the two sequences that are the same when aligned for maximum correspondence over a specified region. As used herein, percentage of sequence identity means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base occurs in both sequences to yield the number of matched positions, dividing the number of matched positions by the total number of positions in the window of comparison and multiplying the result by 100 to yield the percentage of sequence identity.

“Gene” refers to a polynucleotide sequence that comprises control and coding sequences necessary for the production of a polypeptide or precursor. The polypeptide can be encoded by a full length coding sequence or by any portion of the coding sequence. A gene may constitute an uninterrupted coding sequence or it may include one or more introns, bound by the appropriate splice junctions. Moreover, a gene may contain one or more modifications in either the coding or the untranslated regions that could affect the biological activity or the chemical structure of the expression product, the rate of expression, or the manner of expression control. Such modifications include, but are not limited to, mutations, insertions, deletions, and substitutions of one or more nucleotides. In this regard, such modified genes may be referred to as “variants” of the “native” gene.

“Expression” generally refers to the process by which a polynucleotide sequence undergoes successful transcription and translation such that detectable levels of the amino acid sequence or protein are expressed. In certain contexts herein, expression refers to the production of mRNA. In other contexts, expression refers to the production of protein.

“Cell type” refers to a cell from a given source (e.g., tissue or organ) or a cell in a given state of differentiation, or a cell associated with a given pathology or genetic makeup.

“Polypeptide” and “protein”, used interchangeably herein, refer to a polymeric form of amino acids of any length, which may include translated, untranslated, chemically modified, biochemically modified, and derivatized amino acids. A polypeptide or protein may be naturally occurring, recombinant, or synthetic, or any combination of these. Moreover, a polypeptide or protein may comprise a fragment of a naturally occurring protein or peptide. A polypeptide or protein may be a single molecule or may be a multi-molecular complex. In addition, such polypeptides or proteins may have modified peptide backbones. The terms include fusion proteins, including fusion proteins with a heterologous amino acid sequence, fusions with heterologous and homologous leader sequences, with or without N-terminal methionine residues, immunologically tagged proteins, and the like.

A “fragment of a protein” refers to a protein that is a portion of another protein. For example, fragments of proteins may comprise polypeptides obtained by digesting full-length protein isolated from cultured cells. In one embodiment, a protein fragment comprises at least about 6 amino acids. In another embodiment, the fragment comprises at least about 10 amino acids. In yet another embodiment, the protein fragment comprises at least about 16 amino acids.

An “expression product” or “gene product” is a biomolecule, such as a protein or mRNA, that is produced when a gene in an organism is transcribed or translated or post-translationally modified.

“Host cell” refers to a microorganism, a prokaryotic cell, a eukaryotic cell or cell line cultured as a unicellular entity that may be, or has been, used as a recipient for a recombinant vector or other transfer of polynucleotides, and includes the progeny of the original cell that has been transfected. The progeny of a single cell may not necessarily be completely identical in morphology or in genomic or total DNA complement as the original parent due to natural, accidental, or deliberate mutation.

The term “functional equivalent” is intended to include the “fragments”, “mutants”, “derivatives”, “alleles”, “hybrids”, “variants”, “analogs”, or “chemical derivatives” of the native gene or virus.

“Isolated” refers to a polynucleotide, a polypeptide, an immunoglobulin, a virus or a host cell that is in an environment different from that in which the polynucleotide, the polypeptide, the immunoglobulin, the virus or the host cell naturally occurs.

“Substantially purified” refers to a compound that is removed from its natural environment and is at least about 60% free, at least about 65% free, at least about 70% free, at least about 75% free, at least about 80% free, at least about 83% free, at least about 85% free, at least about 88% free, at least about 90% free, at least about 91% free, at least about 92% free, at least about 93% free, at least about 94% free, at least about 95% free, at least about 96% free, at least about 97% free, at least about 98% free, at least about 99% free, at least about 99.9% free, or at least about 99.99% or more free from other components with which it is naturally associated.

“Diagnosis” and “diagnosing” generally includes a determination of a subject's susceptibility to a disease or disorder, a determination as to whether a subject is presently affected by a disease or disorder, a prognosis of a subject affected by a disease or disorder (e.g., identification of pre-metastatic or metastatic cancerous states, stages of cancer, or responsiveness of cancer to therapy), and therametrics (e.g., monitoring a subject's condition to provide information as to the effect or efficacy of therapy).

“Biological sample” encompasses a variety of sample types obtained from an organism that may be used in a diagnostic or monitoring assay. The term encompasses blood and other liquid samples of biological origin, solid tissue samples, such as a biopsy specimen, or tissue cultures or cells derived therefrom and the progeny thereof. The term specifically encompasses a clinical sample, and further includes cells in cell culture, cell supernatants, cell lysates, serum, plasma, urine, amniotic fluid, biological fluids, and tissue samples. The term also encompasses samples that have been manipulated in any way after procurement, such as treatment with reagents, solubilization, or enrichment for certain components.

“Individual”, “subject”, “host” and “patient”, used interchangeably herein, refer to any mammalian subject for whom diagnosis, treatment, or therapy is desired. In one preferred embodiment, the individual, subject, host, or patient is a human. Other subjects may include, but are not limited to, cattle, horses, dogs, cats, guinea pigs, rabbits, rats, primates, and mice.

“Hybridization” refers to any process by which a polynucleotide sequence binds to a complementary sequence through base pairing. Hybridization conditions can be defined by, for example, the concentrations of salt or formamide in the prehybridization and hybridization solutions, or by the hybridization temperature, and are well known in the art. Hybridization can occur under conditions of various stringency.

“Stringent conditions” refers to conditions under which a probe may hybridize to its target polynucleotide sequence, but to no other sequences. Stringent conditions are sequence-dependent (e. g., longer sequences hybridize specifically at higher temperatures). Generally, stringent conditions are selected to be about 5° C. lower than the thermal melting point (Tm) for the specific sequence at a defined ionic strength and pH. The Tm is the temperature (under defined ionic strength, pH, and polynucleotide concentration) at which 50% of the probes complementary to the target sequence hybridize to the target sequence at equilibrium. Typically, stringent conditions will be those in which the salt concentration is at least about 0.01 to about 1.0 M sodium ion concentration (or other salts) at about pH 7.0 to about pH 8.3 and the temperature is at least about 30° C. for short probes (e. g., 10 to 50 nucleotides). Stringent conditions may also be achieved with the addition of destabilizing agents, such as formamide.

“Biomolecule” includes polynucleotides and polypeptides.

“Biological activity” refers to the biological behavior and effects of a protein or peptide. The biological activity of a protein may be affected at the cellular level and the molecular level. For example, the biological activity of a protein may be affected by changes at the molecular level. For example, an antisense oligonucleotide may prevent translation of a particular mRNA, thereby inhibiting the biological activity of the protein encoded by the mRNA. In addition, an immunoglobulin may bind to a particular protein and inhibit that protein's biological activity.

“Oligonucleotide” refers to a polynucleotide sequence comprising, for example, from about 10 nucleotides (nt) to about 1000 nt. Oligonucleotides for use in the invention are for instance from about 15 nt to about 150 nt, for instance from about 150 nt to about 1000 nt in length. The oligonucleotide may be a naturally occurring oligonucleotide or a synthetic oligonucleotide.

“Modified oligonucleotide” and “Modified polynucleotide” refer to oligonucleotides or polynucleotides with one or more chemical modifications at the molecular level of the natural molecular structures of all or any of the bases, sugar moieties, internucleoside phosphate linkages, as well as to molecules having added substitutions or a combination of modifications at these sites. The internucleoside phosphate linkages may be phosphodiester, phosphotriester, phosphoramidate, siloxane, carbonate, carboxymethylester, acetamidate, carbamate, thioether, bridged phosphoramidate, bridged methylene phosphonate, phosphorothioate, methylphosphonate, phosphorodithioate, bridged phosphorothioate or sulfone internucleotide linkages, or 3′-3′, 5′-3′, or 5′-5′linkages, and combinations of such similar linkages. The phosphodiester linkage may be replaced with a substitute linkage, such as phosphorothioate, methylamino, methylphosphonate, phosphoramidate, and guanidine, and the ribose subunit of the polynucleotides may also be substituted (e. g., hexose phosphodiester; peptide nucleic acids). The modifications may be internal (single or repeated) or at the end (s) of the oligonucleotide molecule, and may include additions to the molecule of the internucleoside phosphate linkages, such as deoxyribose and phosphate modifications which cleave or crosslink to the opposite chains or to associated enzymes or other proteins. The terms “modified oligonucleotides” and “modified polynucleotides” also include oligonucleotides or polynucleotides comprising modifications to the sugar moieties (e. g., 3′-substituted ribonucleotides or deoxyribonucleotide monomers), any of which are bound together via 5′ to 3′linkages.

“Biomolecular sequence” or “sequence” refers to all or a portion of a polynucleotide or polypeptide sequence.

The term “detectable” refers to a polynucleotide expression pattern which is detectable via the standard techniques of polymerase chain reaction (PCR), reverse transcriptase- (RT) PCR, differential display, and Northern analyses, which are well known to those of skill in the art. Similarly, polypeptide expression patterns may be “detected” via standard techniques including immunoassays such as Western blots.

A “target gene” refers to a polynucleotide, often derived from a biological sample, to which an oligonucleotide probe is designed to specifically hybridize. It is either the presence or absence of the target polynucleotide that is to be detected, or the amount of the target polynucleotide that is to be quantified. The target polynucleotide has a sequence that is complementary to the polynucleotide sequence of the corresponding probe directed to the target. The target polynucleotide may also refer to the specific subsequence of a larger polynucleotide to which the probe is directed or to the overall sequence (e.g., gene or mRNA) whose expression level it is desired to detect.

A “target protein” refers to a polypeptide, often derived from a biological sample, to which a protein-capture agent specifically hybridizes or binds. It is either the presence or absence of the target protein that is to be detected, or the amount of the target protein that is to be quantified. The target protein has a structure that is recognized by the corresponding protein-capture agent directed to the target. The target protein or amino acid may also refer to the specific substructure of a larger protein to which the protein-capture agent is directed or to the overall structure (e. g., gene or mRNA) whose expression level it is desired to detect.

“Complementary” refers to the topological compatibility or matching together of the interacting surfaces of a probe molecule and its target. The target and its probe can be described as complementary, and furthermore, the contact surface characteristics are complementary to each other. Hybridization or base pairing between nucleotides or nucleic acids, such as, for example, between the two strands of a double-stranded DNA molecule or between an oligonucleotide probe and a target are complementary.

“Label” refers to agents that are capable of providing a detectable signal, either directly or through interaction with one or more additional members of a signal producing system. Labels that are directly detectable and may find use in the invention include fluorescent labels. Specific fluorophores include fluorescein, rhodamine, BODIPY, cyanine dyes and the like.

The term “fusion protein” refers to a protein composed of two or more polypeptides that, although typically not joined in their native state, are joined by their respective amino and carboxyl termini through a peptide linkage to form a single continuous polypeptide. It is understood that the two or more polypeptide components can either be directly joined or indirectly joined through a peptide linker/spacer.

The term “normal physiological conditions” means conditions that are typical inside a living organism or a cell. Although some organs or organisms provide extreme conditions, the intra-organismal and intra-cellular environment normally varies around pH 7 (i.e., from pH 6.5 to pH 7.5), contains water as the predominant solvent, and exists at a temperature above 0° C. and below 50° C. The concentration of various salts depends on the organ, organism, cell, or cellular compartment used as a reference.

“BLAST” refers to Basic Local Alignment Search Tool, a technique for detecting ungapped sub-sequences that match a given query sequence.

“BLASTP” is a BLAST program that compares an amino acid query sequence against a protein sequence database. “BLASTX” is a BLAST program that compares the six-frame conceptual translation products of a nucleotide query sequence (both strands) against a protein sequence database.

A “cds” is used in a GenBank DNA sequence entry to refer to the coding sequence. A coding sequence is a sub-sequence of a DNA sequence that is surmised to encode a gene.

A “consensus” or “contig sequence”, as understood herein, is a group of assembled overlapping sequences, particularly between sequences in one or more of the databases of the invention.

The nucleic acid molecules of the present invention can be produced by a virus harbouring a nucleic acid that encodes the relevant gene sequence. The virus may comprise elements capable of controlling and/or enhancing expression of the nucleic acid. The virus may be a recombinant virus. The recombinant virus may also include other functional elements. For instance, recombinant viruses can be designed such that the viruses will autonomously replicate in the target cell. In this case, elements that induce nucleic acid replication may be required in a recombinant virus. The recombinant virus may also comprise a promoter or regulator or enhancer to control expression of the nucleic acid as required. Tissue specific promoter/enhancer elements may be used to regulate expression of the nucleic acid in specific cell types. The promoter may be constitutive or inducible.

A “promoters” is a region of DNA that is generally located upstream (towards the 5′ region) of the gene that is needed to be transcribed. The promoter permits the proper activation or repression of the gene which it controls. Examples of promoters which are suitable for the invention are the human rhodopsin promoter (Allocca et al., Novel AAV serotypes efficiently transduce murine photoreceptors, J Virol. (2007)), the human red opsin promoter (Nathan et al., Science. 1986 Apr. 11; 232(4747):193-202) or the red cone opsin promoter, the arr3 promoter (Zhu, X. et al. Mouse cone arrestin gene characterization: promoter targets expression to cone photoreceptors. FEBS Letters 524, 116-122 (2002)).

Contaminant components of its natural environment are materials that would interfere with the methods and compositions of the invention, and may include enzymes, hormones, and other proteinaceous or nonproteinaceous solutes. Ordinarily, an isolated agent will be prepared by at least one purification step. In one embodiment, the agent is purified to at least about 60%, at least about 65%, at least about 70%, at least about 75%, at least about 80%, at least about 85%, at least about 88%, at least about 90%, at least about 92%, at least about 95%, at least about 97%, at least about 98%, at least about 99%, at least about 99.9%, or at least about 99.99% by weight.

“Expressing” a protein in a cell means to ensure that the protein is present in the cell, e. g., for the purposes of a procedure of interest. In numerous embodiments, “expressing” a protein will comprise introducing a transgene into a cell comprising a polynucleotide encoding the protein, operably linked to a promoter, wherein the promoter is a constitutive promoter, or an inducible promoter where the conditions sufficient for induction are created, as well as a localization sequence. However, a cell that, e. g., naturally expresses a protein of interest, can be used without manipulation and is considered as “expressing” the protein.

A “fluorescent probe” refers to any compound with the ability to emit light of a certain wavelength when activated by light of another wavelength.

“Fluorescence” refers to any detectable characteristic of a fluorescent signal, including intensity, spectrum, wavelength, intracellular distribution, etc.

“Detecting” fluorescence refers to assessing the fluorescence of a cell using qualitative or quantitative methods. For instance, the fluorescence is determined using quantitative means, e. g., measuring the fluorescence intensity, spectrum, or intracellular distribution, allowing the statistical comparison of values obtained under different conditions. The level can also be determined using qualitative methods, such as the visual analysis and comparison by a human of multiple samples, e. g., samples detected using a fluorescent microscope or other optical detector (e. g., image analysis system, etc.) An “alteration” or “modulation” in fluorescence refers to any detectable difference in the intensity, intracellular distribution, spectrum, wavelength, or other aspect of fluorescence under a particular condition as compared to another condition. For example, an “alteration” or “modulation” is detected quantitatively, and the difference is a statistically significant difference. Any “alterations” or “modulations” in fluorescence can be detected using standard instrumentation, such as a fluorescent microscope, CCD, or any other fluorescent detector, and can be detected using an automated system, such as the integrated systems, or can reflect a subjective detection of an alteration by a human observer.

An assay performed in a “homogeneous format” means that the assay can be performed in a single container, with no manipulation or purification of any components being required to determine the result of the assay, e. g., a test agent can be added to an assay system and any effects directly measured. Often, such “homogeneous format” assays will comprise at least one component that is “quenched” or otherwise modified in the presence or absence of a test agent. ell.

As used herein, “cells” can include whole cells (untreated cells), permeabilized cells, isolated mitochondria, and proteoliposomes, e. g., proteoliposomes reconstituted with a UCP or another protein of interest. The care and maintenance of cells, including yeast cells, is well known to those of skill in the art and can be found in any of a variety of sources, such as Freshney (1994) Culture of Animal Cells. Manual of Basic Technique, Wiley-Liss, New York, Guthrie & Fink (1991), Guthrie and Fink, Guide to Yeast Genetics and Molecular Biology, Academic Press, Ausubel et al. (1999) Current Protocols in Molecular Biology, Greene Publishing Associates, and others.

Cells can be used at any of a wide range of densities, depending on the dye, the test agent, and the particular assay conditions. For instance, a density of about OD₆₀₀=0.01 to 1 is used, for example between about 0.05 and 0.5, e.g. about 0.1.

Expression cassettes are typically introduced into a vector that facilitates entry of the expression cassette into a host cell and maintenance of the expression cassette in the host cell. Such vectors are commonly used and are well know to those of skill in the art. Numerous such vectors are commercially available, e. g., from Invitrogen, Stratagene, Clontech, etc., and are described in numerous guides, such as Ausubel, Guthrie, Strathem, or Berger, all supra. Such vectors typically include promoters, polyadenylation signals, etc. in conjunction with multiple cloning sites, as well as additional elements such as origins of replication, selectable marker genes (e. g., LEU2, URA3, TRP 1, HIS3, GFP), centromeric sequences, etc.

For expression in mammalian cells, any of a number of vectors can be used, such as pSV2, pBC12BI, and p91023, as well as lytic virus vectors (e. g., vaccinia virus, adenovirus, baculovirus), episomal virus vectors (e. g., bovine papillomavirus), and retroviral vectors (e. g., murine retroviruses).

As used herein, the term “disorder” refers to an ailment, disease, illness, clinical condition, or pathological condition.

As used herein, the term “pharmaceutically acceptable carrier” refers to a carrier medium that does not interfere with the effectiveness of the biological activity of the active ingredient, is chemically inert, and is not toxic to the patient to whom it is administered.

As used herein, the term “pharmaceutically acceptable derivative” refers to any homolog, analog, or fragment of an agent, e.g. identified using a method of screening of the invention, that is relatively non-toxic to the subject.

The term “therapeutic agent” refers to any molecule, compound, or treatment, that assists in the prevention or treatment of disorders, or complications of disorders.

Compositions comprising such an agent formulated in a compatible pharmaceutical carrier may be prepared, packaged, and labeled for treatment.

If the complex is water-soluble, then it may be formulated in an appropriate buffer, for example, phosphate buffered saline or other physiologically compatible solutions.

Alternatively, if the resulting complex has poor solubility in aqueous solvents, then it may be formulated with a non-ionic surfactant such as Tween, or polyethylene glycol. Thus, the compounds and their physiologically acceptable solvates may be formulated for administration by inhalation or insufflation (either through the mouth or the nose) or oral, buccal, parenteral, rectal administration or, in the case of tumors, directly injected into a solid tumor.

For administration, the pharmaceutical preparation may be in liquid form, for example, solutions, syrups or suspensions, or may be presented as a drug product for reconstitution with water or other suitable vehicle before use. Such liquid preparations may be prepared by conventional means with pharmaceutically acceptable additives such as suspending agents (e. g., sorbitol syrup, cellulose derivatives or hydrogenated edible fats); emulsifying agents (e. g., lecithin or acacia); non-aqueous vehicles (e. g., almond oil, oily esters, or fractionated vegetable oils); and preservatives (e. g., methyl or propyl-p-hydroxybenzoates or sorbic acid). The pharmaceutical compositions may take the form of, for example, tablets or capsules prepared by conventional means with pharmaceutically acceptable excipients such as binding agents (e. g., pregelatinized maize starch, polyvinyl pyrrolidone or hydroxypropyl methylcellulose); fillers (e. g., lactose, microcrystalline cellulose or calcium hydrogen phosphate); lubricants (e. g., magnesium stearate, talc or silica); disintegrants (e. g., potato starch or sodium starch glycolate); or wetting agents (e. g., sodium lauryl sulphate). The tablets may be coated by methods well-known in the art.

For administration by inhalation, the compounds for use according to the present invention are conveniently delivered in the form of an aerosol spray presentation from pressurized packs or a nebulizer, with the use of a suitable propellant, e. g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, e. g., gelatin for use in an inhaler or insufflator may be formulated containing a powder mix of the compound and a suitable powder base such as lactose or starch.

The compounds may be formulated for parenteral administration by injection, e. g., by bolus injection or continuous infusion. Formulations for injection may be presented in unit dosage form, e. g., in ampoules or in multi-dose containers, with an added preservative.

The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e. g., sterile pyrogen-free water, before use.

The compounds may also be formulated in rectal compositions such as suppositories or retention enemas, e. g., containing conventional suppository bases such as cocoa butter or other glycerides.

The compounds may also be formulated as a topical application, such as a cream or lotion.

In addition to the formulations described previously, the compounds may also be formulated as a depot preparation. Such long acting formulations may be administered by implantation (for example, subcutaneously or intramuscularly) or by intramuscular injection.

Thus, for example, the compounds may be formulated with suitable polymeric or hydrophobic materials (for example, as an emulsion in an acceptable oil) or ion exchange resins, or as sparingly soluble derivatives, for example, as a sparingly soluble salt. Liposomes and emulsions are well known examples of delivery vehicles or carriers for hydrophilic drugs.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration.

The invention also provides kits for carrying out the therapeutic regimens of the invention. Such kits comprise in one or more containers therapeutically or prophylactically effective amounts of the compositions in pharmaceutically acceptable form.

The composition in a vial of a kit may be in the form of a pharmaceutically acceptable solution, e. g., in combination with sterile saline, dextrose solution, or buffered solution, or other pharmaceutically acceptable sterile fluid. Alternatively, the complex may be lyophilized or desiccated; in this instance, the kit optionally further comprises in a container a pharmaceutically acceptable solution (e. g., saline, dextrose solution, etc.), preferably sterile, to reconstitute the complex to form a solution for injection purposes.

In another embodiment, a kit further comprises a needle or syringe, preferably packaged in sterile form, for injecting the complex, and/or a packaged alcohol pad. Instructions are optionally included for administration of compositions by a clinician or by the patient.

A further embodiment of the present invention is a method of screening for agents influencing and/or restoring the formation and growth of the outer segment of photoreceptors, as assessed by e.g. imaging or any other suitable method. In this method of screening, retinal cells generated from stem cells, e.g. IPCs, which do not present outer segments, or not yet present outer segments, are contacted with different agents. Agents inducing the growth of the outer segment on these cells are candidates for the development of a drug for the treatment of ciliopathies and/or photoreceptor dysfunctions.

As used herein, the term “ciliopathies” or “ciliopathy” refers to genetic disorder of the cellular cilia or the cilia anchoring structures, the basal bodies or of ciliary function. Examples of ciliopathies are Alstrom syndrome, Bardet-Biedl syndrome, Joubert syndrome, Meckel-Gruber syndrome, nephronophthisis, orofaciodigital syndrome 1, Senior-Loken syndrome, polycystic kidney disease, primary ciliary dyskinesia, asphyxiating thoracic dysplasia (Jeune), Marden-Walker syndrome, situs inversus/Isomerism, polycystic liver disease, retinal degeneration, agenesis of the corpus callosum, anencephaly, breathing abnormalities, cerebellar vermis hypoplasia, Dandy-Walker malformation, diabetes, Ellis-van Creveld syndrome, exencephaly, eye movement abnormalities, liver disease, hypoplasia of the corpus callosum, hypotonia, reproductive sterility, Jeune asphyxiating thoracic dystrophy, Juvenile myoclonic epilepsy (JME), Marden-Walker syndrome, retinitis pigmentosa, sensorineural deafness, and spina bifida.

As used herein, the term “photoreceptor dysfunction” or “photoreceptor dysfunctions” refers to retinal disorders which lead to a loss of function of photoreceptors. Examples thereof are macular degeneration, retinitis pigmentosa (syndromic and non-syndromic), cone dystrophy, Usher syndrome, rod dystrophy, rod-cone dystrophy, achromatopsia, retina degeneration and age related macular degeneration.

A microRNA, also called. miRNA, is a small non-coding RNA molecule (ca. 22 nucleotides) found in plants and animals, which functions in transcriptional and post-transcriptional regulation of gene expression. Encoded by eukaryotic nuclear DNA, miRNAs function via base-pairing with complementary sequences within mRNA molecules, usually resulting in gene silencing via translational repression or target degradation. miRNAs are well conserved in eukaryotic organisms and are thought to be a vital and evolutionarily ancient component of genetic regulation. While core components of the microRNA pathway are conserved between plants and animals, miRNA repertoires in the two kingdoms appear to have evolved independently with different modes of function. Plant miRNAs usually have perfect or near-perfect pairing with their messenger RNA targets and induce gene repression through degradation of their target transcripts. Plant miRNAs may bind their targets in both coding regions and untranslated regions. In contrast, animal miRNAs typically exhibit only partial complementarity to their mRNA targets. A ‘seed region’ of about 6-8 nucleotides in length at the 5′ end of an animal miRNA is thought to be an important determinant of target specificity. Combinatorial regulation is a feature of miRNA regulation. A given miRNA may have multiple different mRNA targets, and a given target might similarly be targeted by multiple miRNAs. By affecting gene regulation, miRNAs are likely to be involved in most biological processes. Different sets of expressed miRNAs are found in different cell types and tissues.

MicroRNAs are produced from either their own genes or from introns. The majority of the characterized miRNA genes are intergenic or oriented antisense to neighboring genes and are therefore suspected to be transcribed as independent units. However, in some cases a microRNA gene is transcribed together with its host gene; this provides a mean for coupled regulation of miRNA and protein-coding gene. As much as 40% of miRNA genes may lie in the introns of protein and non-protein coding genes or even in exons of long nonprotein-coding transcripts. Other miRNA genes showing a common promoter include the 42-48% of all miRNAs originating from polycistronic units containing multiple discrete loops from which mature miRNAs are processed, although this does not necessarily mean the mature miRNAs of a family will be homologous in structure and function. The promoters mentioned have been shown to have some similarities in their motifs to promoters of other genes transcribed by RNA polymerase II such as protein coding genes.

miRNA genes are usually transcribed by RNA polymerase II (Pol II). The polymerase often binds to a promoter found near the DNA sequence encoding what will become the hairpin loop of the pre-miRNA. The resulting transcript is capped with a specially modified nucleotide at the 5′ end, polyadenylated with multiple adenosines (a poly(A) tail), and spliced. Animal miRNAs are initially transcribed as part of one arm of an ˜80 nucleotide RNA stem-loop that in turn forms part of a several hundred nucleotides long miRNA precursor termed a primary miRNA (pri-miRNA)s. When a stem-loop precursor is found in the 3′ UTR, a transcript may serve as a pri-miRNA and a mRNA. RNA polymerase III (Pol III) transcribes some miRNAs, especially those with upstream Alu sequences, transfer RNAs (tRNAs), and mammalian wide interspersed repeat (MWIR) promoter units. A single pri-miRNA may contain from one to six miRNA precursors. These hairpin loop structures are composed of about 70 nucleotides each. Each hairpin is flanked by sequences necessary for efficient processing. The double-stranded RNA structure of the hairpins in a pri-miRNA is recognized by a nuclear protein known as DiGeorge Syndrome Critical Region 8 (DGCR8 or “Pasha” in invertebrates), named for its association with DiGeorge Syndrome. DGCR8 associates with the enzyme Drosha, a protein that cuts RNA, to form the “Microprocessor” complex. In this complex, DGCR8 orients the catalytic RNase III domain of Drosha to liberate hairpins from pri-miRNAs by cleaving RNA about eleven nucleotides from the hairpin base (two helical RNA turns into the stem). The product resulting has a two-nucleotide overhang at its 3′ end; it has 3′ hydroxyl and 5′ phosphate groups. It is often termed as a pre-miRNA (precursor-miRNA).

Pre-miRNA hairpins are exported from the nucleus in a process involving the nucleocytoplasmic shuttle Exportin-5. This protein, a member of the karyopherin family, recognizes a two-nucleotide overhang left by the RNase III enzyme Drosha at the 3′ end of the pre-miRNA hairpin. Exportin-5-mediated transport to the cytoplasm is energy-dependent, using GTP bound to the Ran protein. In the cytoplasm, the pre-miRNA hairpin is cleaved by the RNase III enzyme Dicer. This endoribonuclease interacts with the 3′ end of the hairpin and cuts away the loop joining the 3′ and 5′ arms, yielding an imperfect miRNA:miRNA* duplex about 22 nucleotides in length. Overall hairpin length and loop size influence the efficiency of Dicer processing, and the imperfect nature of the miRNA:miRNA* pairing also affects cleavage. Although either strand of the duplex may potentially act as a functional miRNA, only one strand is usually incorporated into the RNA-induced silencing complex (RISC) where the miRNA and its mRNA target interact.

The mature miRNA is part of an active RNA-induced silencing complex (RISC) containing Dicer and many associated proteins. RISC is also known as a microRNA ribonucleoprotein complex (miRNP); RISC with incorporated miRNA is sometimes referred to as “miRISC.”

Dicer processing of the pre-miRNA is thought to be coupled with unwinding of the duplex. Generally, only one strand is incorporated into the miRISC, selected on the basis of its thermodynamic instability and weaker base-pairing relative to the other strand. The position of the stem-loop may also influence strand choice. The other strand, called the passenger strand due to its lower levels in the steady state, is denoted with an asterisk (*) and is normally degraded. In some cases, both strands of the duplex are viable and become functional miRNA that target different mRNA populations.

Members of the Argonaute (Ago) protein family are central to RISC function. Argonautes are needed for miRNA-induced silencing and contain two conserved RNA binding domains: a PAZ domain that can bind the single stranded 3′ end of the mature miRNA and a PIWI domain that structurally resembles ribonuclease-H and functions to interact with the 5′ end of the guide strand. They bind the mature miRNA and orient it for interaction with a target mRNA. Some argonautes, for example human Ago2, cleave target transcripts directly; argonautes may also recruit additional proteins to achieve translational repression. The human genome encodes eight argonaute proteins divided by sequence similarities into two families: AGO (with four members present in all mammalian cells and called E1F2C/hAgo in humans), and PIWI (found in the germ line and hematopoietic stem cells).

Additional RISC components include TRBP [human immunodeficiency virus (HIV) transactivating response RNA (TAR) binding protein], PACT (protein activator of the interferon induced protein kinase (PACT), the SMN complex, fragile X mental retardation protein (FMRP), Tudor staphylococcal nuclease-domain-containing protein (Tudor-SN), the putative DNA helicase MOV10, and the RNA recognition motif containing protein TNRC6B.

Gene silencing may occur either via mRNA degradation or preventing mRNA from being translated.

miRNA-182:
(SEQ ID NO: 1)
uuuggcaaugguagaacucacacu
and
passenger strand
(SEQ ID NO: 2)
ugguucuagacuugccaacua.
miRNA-183:
(SEQ ID NO: 3)
uauggcacugguagaauucacu
and
passenger strand
(SEQ ID NO: 4)
gugaauuaccgaagggccauaa.
miRNA-96:
(SEQ ID NO: 5)
uuuggcacuagcacauuuuugcu
and
passenger strand
(SEQ ID NO: 6)
aaucaugugcagugccaauaug.

All three miRNAs (miRNA-182, miRNA-96 and miRNA-183) are present in the genome as a cluster.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the present specification, including definitions, will control. In addition, the materials, methods, and examples are illustrative only and not intended to be limiting.

### Examples

### Materials and Methods

### Animals.

All animal experiments and procedures were approved by the Swiss Veterinary Office. The animals were maintained under a 12-hour light-dark cycle. Conditional DGCR8 knockout mice (R. Yi et al., DGCR8-dependent microRNA biogenesis is essential for skin development, Proc. Natl. Acad. Sci. U.S.A. 106, 498-502 (2009)) were in-house crossed to the cone photoreceptor-specific Cre recombinase driver line D4-cre (Y.-Z. Le et al., Targeted expression of Cre recombinase to cone photoreceptors in transgenic mice, Mol. Vis. 10, 1011-1018 (2004)) and the floxed tdTomato reporter line Ai9 (JAX mice B6.Cg-Gt(ROSA)26Sor^{tm9(CAG-tdTomato)Hze}/J, Stock Number 007909, Jackson Laboratory), resulting in C-DGCR-KO animals. D4-cre or D4-cre/Ai9 animals served as wild type controls.

### ES Cell-Derived Retina-Like Structure Formation.

Rx-GFP K/I EB5 ES cells (M. Eiraku et al., Self-organizing optic-cup morphogenesis in three-dimensional culture, Nature 472, 51-56 (2011)) (RIKEN Cell Bank, Ibaraki, Japan), derived from mixed 129-C57BL/6 background mice, were maintained in medium containing GMEM, 1% FBS, 10% knockout serum replacement (KSR), 0.1 mM NEAA, 1 mM sodium pyruvate, 2000 U/ml mouse LIF, and 0.1 mM 2-mercaptoethanol. Differentiation was performed essentially as described previously (M. Eiraku et al., Self-organizing optic-cup morphogenesis in three-dimensional culture, Nature 472, 51-56 (2011); M. Eiraku, Y. Sasai, Mouse embryonic stem cell culture for generation of three-dimensional retinal and cortical tissues, Nat Protoc 7, 69-79 (2012)). Briefly, ES cells were dissociated to single cells by 0.25% trypsin-EDTA treatment and re-aggregated in differentiation medium (GMEM, 1.5% KSR, 0.1 mM NEAA, 1 mM pyruvate, 1 mM 2-mercaptoethanol) at a density of 3,000 cells per 100 ml per well of 96-well low-cell-adhesion plates (Lipidure Coat, NOF). The matrigel (growth-factor-reduced; BD Biosciences) was added to culture to final 2% (v/v) 24 h later. Cell aggregates were transferred to 35×10 mm EASY-GRIP dishes (Polystiren) on day 7 and further cultured in suspension in DMEM/F12 medium supplemented with the N2 additive under 40% O₂/5% CO₂ conditions. On day 11, Rx-GFP-positive optic cups were mechanically separated from aggregates and further cultured in medium containing DMEM/F12, N2, 10% FBS, 1 mM taurine, and 0.5 μM all-trans retinoic acid for the next 15-20 days. Self-complementary (D. M. McCarty, P. E. Monahan, R. J. Samulski, Self-complementary recombinant adeno-associated virus (scAAV) vectors promote efficient transduction independently of DNA synthesis, Gene Ther. 8, 1248-1254 (2001)) AAV-expressing GFP and either engineered pri-miR-183/96/182 transcript or control RNA was added to the culture on day 7.

### AAV Production.

To generate pAAV2-CMV-DIO-DGCR8, the present inventors first in silico designed a DGCR8 insert, then synthesized the insert by DNA2.0 Inc. (Menlo Park, Calif. 94025, USA), and finally ligated (Mighty Mix #6023 by TaKaRa) the insert into pAAV2-Rho-EGFP (M. Allocca et al., Novel adeno-associated virus serotypes efficiently transduce murine photoreceptors, J. Virol. 81, 11372-11380 (2007)) cut by NheI/HindIII (New England Biolabs). To create the insert, mouse DGCR8 cDNA (NCBI Reference Sequence: NM_033324.2; residues 347-2668) was modified to include an optimized Kozak sequence (GCCACCATG), inserted in inverted orientation into CMV promoter DIO (double-floxed inverted open reading frame) expression cassette and the cassette was equipped with 5′-NheI and 3′-HindIII adapters. AAV production was performed as previously described (J. C. Grieger, V. W. Choi, R. J. Samulski, Production and characterization of adeno-associated viral vectors, Nat Protoc 1, 1412-1428 (2006)) by triple transfection of HEK293T cells with branched polyethylenimine (Polysciences, no. 23966) with a plasmid containing the transgene between the internal terminal repeats of AAV2, the AAV-helper plasmid encoding Rep2 and Cap for serotype 8, and the pHGTI-Adenol plasmid harboring helper adenoviral genes (both kindly provided by C. Cepko, Harvard Medical School, Boston, Mass., USA). Vectors were purified using a discontinuous iodixanol gradient (Sigma, Optiprep). Encapsidated DNA was quantified by TaqMan RT-PCR following denaturation of the AAV particles using ProteinaseK, and titers were calculated as genome copies (GC) per ml.

### Subretinal AAV Delivery.

The injection of viral particles was performed as previously described (V. Busskamp et al., Genetic reactivation of cone photoreceptors restores visual responses in retinitis pigmentosa, Science 329, 413-417 (2010)). Briefly, animals were anesthetized using 3% isoflurane. A small incision was made with a sharp 30-gauge needle in the sclera near the lens. 2 μl of AAV suspension was injected through this incision into the subretinal space using a blunt 5-μl Hamilton syringe held in a micromanipulator.

Conditional Expression of miR-182/183 or miR-124 Mimics.

Short hairpin (sh) RNAs resembling pre-miR-182, -183 and -124 were designed according to Dicer properties of the pre-miRNA cleavage (J. Krol et al., Structural features of microRNA (miRNA) precursors and their relevance to miRNA biogenesis and small interfering RNA/short hairpin RNA design, J. Biol. Chem. 279, 42230-42239 (2004); J. Starega-Roslan et al., Structural basis of microRNA length variety, Nucleic Acids Res. 39, 257-268 (2011)) to allow the generation of mature miR-182-5p, miR-183-5p or miR-124 sequences, as annotated in miRBASE v.20 (www.mirbase.org). Conditional expression of shRNA miRNA mimics (sh-miRs) was driven by H1-TetO2 promoter and was inhibited in Cre(-) cells by Tet repressor (TetR) (W. Hillen, C. Berens, Mechanisms underlying expression of Tn10 encoded tetracycline resistance, Annu. Rev. Microbiol. 48, 345-369 (1994)). Sequences of H1-TetO2 promoter and TetR were taken from pSUPERIOR.neo (OligoEngine) and pcDNA6/TR (Invitrogen) plasmids, respectively. Following in silico design, DNA fragments containing TetR and inverted EGFP sequences were inserted into the CMV promoter DIO expression cassette, followed by the H1-TetO2 promoter driving sh-miR or sh-Control sequences. This construct was synthesized by GENEWIZ Inc. (South Plainfield, N.J. 07080, USA) and cloned into pAAV2 vectors. Expression of mature miRNA mimics was verified by RT-qPCR, using RNA isolated from AAV-infected HEK293 cells grown in the presence of tetracycline to inhibit TetR.

### Reporter Luciferase Assays.

A reporter assay was used to verify functionality of miRNA mimics in HEK293 cells. Firefly luciferase (pFL) and Renilla luciferase (pRL) vectors were made as previously described (J. Krol et al., Characterizing light-regulated retinal microRNAs reveals rapid turnover as a common property of neuronal microRNAs, Cell 141, 618-631 (2010); R. S. Pillai et al., Inhibition of translational initiation by Let-7 MicroRNA in human cells, Science 309, 1573-1576 (2005)). MiRNA-binding sequences specific for mouse miR-182/183 or miR-124 were designed to have four miRNA binding sites for each individual miRNA, interrupted by 15-nt spacers. Binding sites for miRNAs were perfectly complementary to the seed and 3′-proximal miRNA region, with a bulge at positions 9-12 to prevent cleavage of reporter RNA. The DNA fragments harboring miRNA-binding regions were synthesized by GENEWIZ Inc., and cloned into an XbaI site downstream of the FL ORF in pFL. Sequencing verified that all plasmids were correct. HEK293 cells were cotransfected with pFL reporters, control pRL plasmid, and an AAV vector expressing sh-miR in the presence (1 μg/ml) or absence of tetracycline. Tetracycline was used to block TetR repressor activity and allow H1-TetO2-driven expression of sh-miRs. Cell lysates were prepared using Passive Lysis Buffer (Promega) 48 h after transfection, and luciferase activities were measured using the Dual Luciferase Reporter Assay (Promega).

### Quantitative Western Blots.

Isolated cones from C-DGCR-KO or wild type mice were lysed on ice in a buffer containing 50 mM Tris pH 7.5, 10 mM DTT, 10 mM EDTA, 1% SDS, and 1× complete protease inhibitor cocktail (Roche). Protein lysates (15 μg/lane) were separated by SDS-PAGE followed by electrotransfer to PVDF membrane (Millipore). Membranes were incubated for 2 h at room temperature with primary antibodies: rabbit-anti-DGCR8 (1:200, Abcam), goat-anti-OPN1MW (1:300, Milipore), goat-anti-OPN1SW (1:1,000, Milipore), rabbit-anti-Dicer D347 (E. Billy, V. Brondani, H. Zhang, U. Müller, W. Filipowicz, Specific interference with gene expression induced by long, double-stranded RNA in mouse embryonal teratocarcinoma cell lines, Proc. Natl. Acad. Sci. U.S.A. 98, 14428-14433 (2001)) (1:500), mouse-anti-Ago2 (1:200, WAKO), or mouse-anti-β-tubulin (1:10,000, Sigma), and then with peroxidase-conjugated secondary antibodies. For quantitative westerns, IRDye® 680 or IRDye® 800CW secondary antibodies were used and membranes were analyzed with the Odyssey Infrared Imaging System (LI-COR Biosciences). Protein ladder PageRuler™ Plus (Fermentas) was used as a protein size marker.

### Immunohistochemistry.

Immunohistochemistry was performed as previously described (V. Busskamp et al., Genetic reactivation of cone photoreceptors restores visual responses in retinitis pigmentosa, Science 329, 413-417 (2010)). Briefly, retinas of mice or grown from ES cells were isolated and fixed in 4% paraformaldehyde in phosphate-buffered saline (PBS) for 20 min and washed in PBS. Retinal wholemounts or 3% agarose-embedded (SeaKem Le Agarose, Lonza) 100-μm thick vibratome sections (Leica VT1000S vibratome) were incubated in blocking solution (10% normal donkey serum (NDS, Chemicon), 1% bovine serum albumin (BSA), and 0.5% Triton X-100 in PBS, pH 7.4) for 1 h. Primary and secondary antibody applications were done in 3% NDS, 1% BSA, 0.02% sodium azide, and 0.5% Triton X-100 in PBS. Primary antibodies were applied for ˜4 days. Specimens were mounted on a glass slide with ProLong Gold antifade reagent (Invitrogen). As primary antibodies we used goat-anti-OPN1SW (1:200, Santa Cruz, sc-14365), goat-anti-OPN1MW/LW (1:200, Santa Cruz, sc-22117), rabbit-anti-DGCR8 (1:200, Bethyl, A302-469A), sheep-anti-CHX10 (1:400, Exalpha, X2398M), rabbit-anti-Recoverin (1:400, Chemicon, AB5585), and mouse-anti-Rhodopsin (1:800, Sigma, 5403). As secondary antibodies, donkey-anti-goat-Alexa488 (1:200, Invitrogen, A11055), donkey-anti-rabbit-Alexa647 (1:200, Invitrogen, A315773), donkey-anti-sheep-Alexa488 (1:200, Invitrogen, A11015), donkey-anti-rabbit-Alexa568 (1:200, Invitrogen, A10042), and donkey-anti-mouse-Alexa647 (1:200, Invitrogen, A31571) were used. Nuclei were stained with the dye Hoechst (1:600, Invitrogen, H3570, 10 mg/ml). TdTomato signal persisted fixation and staining steps, and it was imaged using the life signal.

### RNA Isolation.

Total RNA from isolated cones or wholemount retina was extracted with Arcturus PicoPure RNA Isolation Kit (Applied Biosystems, Foster City, Calif.) or Trizol, and quantitative and qualitative analysis was performed using a 2100 Bioanalyzer (Agilent Technologies Inc., Santa Clara, Calif., USA).

RT-qPCR Quantification of Mature miRNAs, miRNA Precursors, and mRNAs.

Total RNA from isolated cones was extracted using Trizol. Analysis of mature miRNA levels was performed using the Applied Biosystem Taqman® microRNA Assay System (Applied Biosystems, Foster City, Calif.), as previously described (J. Krol et al., Characterizing light-regulated retinal microRNAs reveals rapid turnover as a common property of neuronal microRNAs, Cell 141, 618-631 (2010)). Briefly, reverse transcription (RT) reactions containing 5 ng RNA, 1×RT buffer, 0.25 mM each dNTP, 0.25 U/μl RNase inhibitor, 3.33 U/μl MultiScribe RT, and 50 nM miRNA-specific RT primer were incubated for 30 min at 16° C. and 30 min at 42° C. The 10-μl PCR reactions contained 0.67 μl of RT reaction, 1× Taqman Universal PCR master mix, and 1 μl of primers and a probe mix of the Taqman® MicroRNA Assay. The reactions were incubated in a 48-well optical plate at 95° C. for 5 min, followed by 40 cycles of 95° C. for 15 s and 60° C. for 60 s. The threshold cycle (Ct) values were determined using default threshold settings. Each reaction was performed in triplicate and the data normalized to U6 snRNA. The expression fold change was calculated as 2^{-ΔΔCt}. For miRNA precursors and mRNA expression analysis, total RNA, treated with RNase-free DNase I, was reverse-transcribed using random hexamers and the Superscript III thermostable RT system according to the manufacturer's instructions (Invitrogen). RT-qPCR was performed using the Applied Biosystems StepOne System using standard protocol with 1/10 diluted cDNA, SYBR green PCR master mix (Applied Biosystems) and 0.5 μM primers. Reactions in triplicate were normalized to 18S rRNA.

### Confocal Microscopy.

Zeiss LSM 700 laser scanning confocal microscope was used to acquire images of antibody-stained retinas with an EC Plan-Neofluar 40×/1.30 oil M27 objective. Morphologies of the cones were assessed from 1024×1024 pixel images in a z-stack with 0.1 μm z-steps. Images were processed using Imaris (Bitplane) or Fiji.

### Serial Block-Face Scanning Electron Microscopy.

Retinas were fixed with 2% paraformaldehyde and 2% glutaraldehyde in 0.1 M Na-cacodylate buffer, pH 7.4, and embedded in 4% agarose. Retinas were cut in 60 μm coronal vibratome sections in PBS and processed according to a modified version of the NCMIR protocol (ncmir.ucsd.edu/sbfsem-protocol.pdf). Sections were collected and rinsed 3×5 min with 0.1 M Na-cacodylate buffer, pH 7.4, and post-fixed with 1.5% potassium ferrocyanide and 1% osmium tetroxide in 0.1M Na-cacodylate buffer for 30 min. After rinsing in ddH₂O, sections were stained in 1% thiocarbohydrazide for 10 min. After rinsing, sections were immersed in 1% osmium tetroxide for 20 min. Following extensive rinsing in ddH₂O, sections were stained en bloc with 1% aqueous uranyl acetate overnight at 4° C. The following day, sections were dehydrated with ethanol and flat-embedded in Epon resin (Serva). After 24 h of curing in an oven at 60° C., sections were screened under a light microscope and the region of interest (ROI) selected. The ROI was cut out of the flat-embedded section and mounted perpendicularly on a pin suited for serial block-face scanning electron microscope (3View from GATAN in an FEI QUANTA 200 VP_FEG scanning electron microscope) in order to have the cones and rods at the top of the block. The tissue was then trimmed and placed in the microscope. The surface of the block was imaged (3.5 kV, spot size 3, 4000×4000 pixels, 12 nm/pixel, 5 ms dwell time) and then 80 or 100 nm was shaved from the surface using a diamond knife before the new surface was imaged. Stacks of 700-1200 images were acquired. Images were then exported in TIF format and registered (translation-rotation) using the TrackEM2 registration procedure. The final images were then exported in TIF format.

### Image Analysis and Quantification.

Confocal microscopy images were quantified by using the automatic cell-counting feature (Spots) in Imaris 7.6 (Bitplane). The number of cone photoreceptors was determined by counting tdTomato-positive or peanut agglutinin (PNA)-lectin-Alexa Fluor® 568 conjugate (Molecular Probes, L-32458) labeled cells. Electron microscope image stacks were analyzed using Fiji software. The EM images were 12 nm (x)×12 nm (y)×100 nm (depth). The length of the cone outer segments was defined by the length of the electrodense material in the outer segment representing the disk membrane.

### Electroretinogram Measurements.

Photopic electroretinograms were measured as previously described (N. Tanimoto, V. Sothilingam, M. W. Seeliger, Functional phenotyping of mouse models with ERG, Methods Mol. Biol. 935, 69-78 (2013)).

### Patch-Clamping Recordings.

Retinas from 2 h dark-adapted mice were isolated and used for patch-clamping recordings as previously described (K. Farrow et al., Ambient illumination toggles a neuronal circuit switch in the retina and visual perception at cone threshold, Neuron 78, 325-338 (2013)). Briefly, electrophysiological recordings were made using an Axon Multiclamp 700B amplifier (Molecular Devices) and borosilicate glass electrodes (Sutter Instrument). Signals were digitized at 10 kHz (National Instruments) and acquired using software written in LabVIEW (National Instruments). Data were analyzed offline using MATLAB (MathWorks). The spiking responses of retinal ganglion cells were recorded using the patch-clamp technique in loose cell-attached mode with electrodes pulled to 3-5MΩ resistance and filled with Ringer's medium.

### Fluorescence-Activated Cell Sorting (FACS).

Retinas were isolated and dissociated to single cells by papain digestion as previously described (J. M. Trimarchi et al., Molecular heterogeneity of developing retinal ganglion and amacrine cells revealed through single cell gene expression profiling, J. Comp. Neurol. 502, 1047-1065 (2007)). Cells positive for tdTomato were sorted using FACS (MoFlo from DakoCytomation) and used for genomic and western blot analysis.

### Affymetrix Microarray Analysis.

Gene expression in isolated cones originating from P30, P40, P50, P60, and P90 C-DGCR-KO or wild type mice was assessed using Affymetrix GeneChip® Mouse Gene 1.0 ST Arrays. For each time point and condition (C-DGCR-KO and wild type) we prepared two independent samples. Analysis was performed at the Functional Genomics Facility of the Friedrich Miescher Institute for Biomedical Research. Total RNA (100 ng per sample) was reverse transcribed and amplified using the Ambion WT Expression kit (Ambion). The sense-strand cDNA obtained was fragmented and labeled using the Affymetrix GeneChip WT Terminal Labeling kit (Affymetrix). Arrays were hybridized for 16 h following the “GeneChip Whole Transcript (WT) Sense Target Labeling Assay Manual” (Affymetrix). The Affymetrix Fluidiscs protocol FS450_0007 was used for washing. Scanning was performed using the Affymetrix GCC Scan Control v. 3.0.0.1214 on a GeneChip Scanner 3000 with autoloader.

### RNA Sequencing.

Gene expression in isolated cones originating from P30, P40, P50, P60, and P90 C-DGCR-KO or wild type mice was assessed using next generation RNA sequencing. For each time point and condition (C-DGCR-KO and wild type) the present inventors prepared two independent samples. Total RNA from isolated cones (150 ng) was processed using the ScripSet Complete (Human/Mouse/Rat) low input kit according to the manufacturer's instructions (Epicentre/Illumina). Libraries were pooled equimolarly in batches of 4, and 12 pM of each pool were sequenced on one lane of the HiSeq 2000 instrument using RTA 1.13.48. Individual reads were assigned to their sample based on the TruSeq barcode using the Illumina software Casava v1.8.0.

### MiRNA Sequencing.

Small RNA (15-30 nt in length) libraries were prepared from 1 μg of total RNA using TruSeq Small RNA Sample Prep Kit according to the manufacturer's protocol (Illumina, San Diego, Calif., USA). Four independent samples at P60, two from wild type and two from C-DGCR-KO mice, were multiplexed and 13 pM of the multiplexed libraries were sequenced on one lane of the HiSeq 2000 instrument using RTA 1.13.48. Each sample was prepared from FACS sorted cones from 20 retinas. Individual reads were assigned to their sample based on the TruSeq barcode using the Illumina software Casava v1.8.0. The 3′ adapter sequence was removed by aligning it to the read, allowing one or two mismatches in prefix alignments of at least 7 or 10 bases, respectively. Low complexity reads were filtered out based on their dinucleotide entropy. All reads shorter than 14 nt were removed.

### MiRNA Target Prediction.

MiRNA target mRNAs were predicted using TargetScan v.6.2 (targetscan.org).

### Selection of Housekeeping Genes.

Critical to the outlined data processing methods is the accurate identification of normalizing housekeeping genes in order to accurately estimate relative gene expression levels. To do this, a list of potential housekeeping genes was identified (Ldha, Actb, Rp119, B2m, Gapdh, Pgk1, Tubbi, Cltc, Tbp) by choosing genes presumed to remain unchanged throughout the time course and operating in independent cellular pathways. Raw intensity time courses for these selected genes were then compared. Those that behaved similarly were accepted as suitable housekeeping genes and those that were outliers were discarded from the list. In the subsequent data processing, gene intensities were normalized to various housekeeping genes to verify relative expression estimates. In some examples, lactate dehydrogenase A (Ldha) was used for housekeeping normalization, as this gene was not a direct target of the miRNAs knocked down in cone cells, and it consistently remained unchanged under the housekeeping selection criteria outlined above.

### Microarray Data Replicate Correction, Summarization and Normalization.

Raw fluorescence intensity CEL files were obtained from the microarray experiment. Three methods were developed to combine data from the two biological replicates taken at each time point-condition combination (e.g. P30 DGCR-KO mouse). In the first method, replicate raw intensities were mapped from one plate to the other using a polynomial fit function. When possible, a cubic polynomial was used, and in cases where such a function could not be fit (because of fewer probes per gene), a lower degree polynomial mapping function was used. This mapping was done probe-wise, i.e., a polynomial was fit individually to each probe set for a given gene. After mapping, the average of the two replicates was taken. For a given gene, the mean or median of the probe set was then used to summarize the gene's raw intensity value. Since intensity levels should correlate with expression levels in the Affymetrix chip, probe set intensities relative to a constant reference level give an estimation of relative gene expression. Summarized gene intensities were normalized to housekeeping gene intensities to provide relative expression estimations comparable across all genes on the chip. In the second method, raw intensities were first summarized by taking the mean or median of the probe set intensities. These values were then normalized to housekeeping gene intensities, followed by averaging across the two biological replicates. The first and second methods described here produced relative gene expression estimations, which could then be used to compare gene expression across conditions and time points in the data analysis. In a variation of the processing approach, the third method summarized gene probe sets at a later stage. In this method the polynomial mapping and replicate averaging was done exactly as in the first method described. As we were interested in changes in gene expression over time, the raw intensities of a given gene were then normalized to the starting time point (in this case, P30) for all genes. These relative intensity values of each probe in the probe set were then summarized by taking the mean or median. Normalization to relative housekeeping intensity values produced an estimation of the relative change in expression over the time course. Each of the three methods performed similarly, yielding nearly identical results. The present inventors used the mean of the two independent samples as data for further analysis.

### RNA Sequencing Data Replicate Correction and Normalization.

RNA sequencing reads, obtained for the wild type and C-DGCR-KO conditions at each time point, were aligned to the mm10 mouse genome assembly using the QuasR Bioconductor package (http://www.bioconductor.org/packages/2.12/bioc/html/QuasR.html) and SpliceMap (K. F. Au, H. Jiang, L. Lin, Y. Xing, W. H. Wong, Detection of splice junctions from paired-end RNA-seq data by SpliceMap, Nucleic Acids Res. 38, 4570-4578 (2010)) from the Rbowtie package (http://www.bioconductor.org/packages/2.12/bioc/html/Rbowtie.html) with default parameters except for “splicedAlignment=TRUE”. A table with the number of alignments per gene was produced by counting alignments starting in annotated exons on the same strand of a gene using the QuasR's qCount function and gene models from the TxDb.Mmusculus.UCSC.mm10.knownGene Bioconductor package (version 2.9.0). Counts were first normalized by the length of the gene locus. An approach similar to second method outlined for the microarray analysis was then used to normalize raw count levels to housekeeping genes, producing relative expression estimates that could then be analyzed for differential expression in the same way as the processed microarray data. We used the mean of the two independent samples as data for further analysis.

### Expression Time Course Analysis and Significance Testing.

Differential gene expression was assessed within each condition (C-DGCR-KO or wild type) over time, comparing expression levels relative to the starting level at P30, as well as between conditions at each time point, comparing the C-DGCR-KO to the wild type. Within each condition, fold changes in expression were calculated by dividing expression estimates at each time point by the P30 estimate. Across conditions, fold changes were calculated by dividing the expression estimate of the knock out by the wild type. To normalize the starting value to one in the comparison across conditions, this relative change was then normalized by the first time point. Significance in time course expression changes was assigned to those genes whose fold change lay outside the 95th percentile at both P60 and P90 relative to P30. A bootstrapping method was used to assess the significance of group up or down regulation. For a family of n genes, n random genes were iteratively selected from the population of genes and treated as a pseudo-family. For each pseudo-family, a summary statistic was calculated, either the mean or median. One million iterations were done to form a distribution, against which the n-gene family of interest could be compared and assigned significance by calculation of the p-value. The cone-specific genes were defined using the gene expression atlas of adult retinal cell types (S. Siegert et al., Transcriptional code and disease map for adult retinal cell types, Nat. Neurosci. 15, 487-495, S1-2 (2012)). A gene was defined cone-specific if its mean expression in cones was more than two times of the maximum expression in other retinal cell types.

### Gene Ontology Term Enrichment Analysis.

Genes that change over time in the C-DGCR-KO samples were identified using edgeR (D. J. McCarthy, Y. Chen, G. K. Smyth, Differential expression analysis of multifactor RNA-Seq experiments with respect to biological variation, Nucleic Acids Res. 40, 4288-4297 (2012)) as follows: Raw alignment counts per gene were scaled between samples and genes with less than five counts per million in at least two samples were removed, retaining a total number of 13289 genes (57.6%). Differential genes with an FDR of less than 1e-6 and a minimal fold-change of two were selected based on the ANOVA-like test, resulting in 333 and 446 significantly up- and down-regulated genes, respectively. Enriched gene ontology terms in the “Biological process” graph were identified using GOstats (S. Falcon, R. Gentleman, Using GOstats to test gene lists for GO term association, Bioinformatics 23, 257-258 (2007)) and GO annotation in org.Mm.eg.db (version 2.9.0), with P values smaller than 0.01 conditional on the GO graph structure. Significant terms with between five and 1000 annotated genes were used for visualization.

### Statistical Analysis.

The non-parametric Mann-Whitney U test was used to compare data.

Significance levels are indicated by * for p<0.05, ** for p<0.01, and *** for p<0.001. n.s. (not significant) means p>0.05. The error bars and ±values represent s.e.m.

To deplete miRNAs from adult cones, the present inventors genetically disrupted the Drosha/DGCR8 miRNA-processing machinery by crossing mice with conditional null Dgcr8 alleles (R. Yi et al., DGCR8-dependent microRNA biogenesis is essential for skin development, Proc. Natl. Acad. Sci. U.S.A. 106, 498-502 (2009)) and mice expressing Cre recombinase postnatally only in cones (Y.-Z. Le et al., Targeted expression of Cre recombinase to cone photoreceptors in transgenic mice, Mol. Vis. 10, 1011-1018 (2004)) (C-DGCR-KO mice). The morphology of cones in C-DGCR-KO mice was revealed by conditional fluorescent protein-expressing reporter mouse lines or conditional adeno-associated viral vectors (AAVs). Cones were examined in retinal sections, in retinal wholemounts, and in isolation after fluorescence-activated cell sorting. At a time when the retina is fully developed, at postnatal day 30 (P30), immunohistochemistry revealed no appreciable difference in DGCR8 signal between C-DGCR-KO and wild type cone nuclei. Quantitative western blot analysis of isolated cones showed 43% DGCR8 levels compared to wild type, but the levels of mature miRNAs decreased by only 20-25%. The morphology of cones and opsin-labeled (OPN1SW and OPN1MW) outer segment distribution in intact retinas assayed by immunohistochemistry, the opsin mRNA and protein levels in isolated cones tested by qPCR and western blot, and the function of cones tested in vivo using electroretinography were similar between C-DGCR-KO and wild type mice. The observation that adult cones of C-DGCR-KO mice at P30 had nearly normal mature miRNA levels, as well as normal structure and function, allowed the inventors to investigate the effect of progressive loss of mature miRNAs in adult cones.

In contrast to P30, the DGCR8 signal in cone nuclei was not detectable on retinal sections at P60 and amounted to only 18% of the wild type in western blot of isolated cones. Moreover, miRNA levels in isolated P60 C-DGCR-KO cones, tested by qPCR, were 95% lower than in wild type cones. Hence, at P60 the DGCR8 function was largely lost and consequently miRNAs were depleted in cones. Remarkably, in this miRNA-depleted cone state the inventors found that the number of cone outer segments labeled with cones opsins was reduced by 90%. Opsin mRNA and protein expression in isolated cones was also markedly reduced. The loss of opsin staining was not due to the death of cones, since the number of cones counted in wholemount retinas was similar in C-DGCR-KO and wild type mice. Since the outer segments and opsin expression are necessary for light detection, the inventors tested the ability of the P60 C-DGCR-KO retina to respond to light stimuli in vivo and ex vivo. In vivo, cone-mediated photoresponses were strongly reduced compared to wild type and P30 C-DGCR-KO retinas. Similar to in vivo recordings, ex vivo single ganglion cell responses measured by using whole-cell patch clamp in high light levels, reflecting cone vision, were significantly reduced. Therefore, by P60 the majority of cones from C-DGCR-KO mice had lost their outer segments and become non-functional, but had not died. The observed visual phenotype presents the clinical picture of achromatopsia (M. Michaelides, D. M. Hunt, A. T. Moore, The cone dysfunction syndromes, Br J Ophthalmol 88, 291-297 (2004)).

To reveal fine morphological changes in the P60 opsin-less cones compared to P30 C-DGCR-KO and wild type cones, the present inventors reconstructed 50×50×170 μm² cubes of outer retina of P30 and P60 C-DGCR-KO and of P60 wild type mice using serial block-face scanning electron microscopy (W. Denk, H. Horstmann, Serial block-face scanning electron microscopy to reconstruct three-dimensional tissue nanostructure, PLoS Biol. 2, e329 (2004)) that allowed them to visualize cones and rods at the ultrastructural level in 3D. Cones could be distinguished from rods based on a number of criteria (L. D. Carter-Dawson, M. M. LaVail, Rods and cones in the mouse retina. I. Structural analysis using light and electron microscopy, J. Comp. Neurol. 188, 245-262 (1979)), the most robust being the organization of heterochromatin in the nucleus (I. Solovei et al., Nuclear architecture of rod photoreceptor cells adapts to vision in mammalian evolution, Cell 137, 356-368 (2009)). Remarkably, all P60 C-DGCR-KO cones lacked outer segments; the inner segments often terminated with a single cilium appearing as a flagless flag holder. Inner segments and mitochondria were larger than in wild type. The cone cell bodies and nuclear organization were intact and did not appear to be different from wild type. Cone morphology in P30 C-DGCR-KO mice and rod photoreceptor morphology in both P30 and P60 C-DGCR-KO mice appeared normal, with the exception of enlarged mitochondria in P30 C-DGCR-KO cones. To determine the time course of outer segment shortening, the inventors 3D-reconstructed the outer retina in ten-day intervals between P30 and P60. Cone outer segments shortened in a linear fashion from P30 to P60.

To test if the loss of opsin can be prevented when outer segments are shortening, the inventors reintroduced Dgcr8 to P45 C-DGCR-KO cones via conditional AAV-mediated delivery. At P90, DGCR8 and opsin protein levels in cones were significantly higher in AAV-infected C-DGCR-KO retinas compared to uninfected control retinas. The lack of DGCR8 could cause defects either through miRNA-dependent or independent pathways (D. G. Ryan, M. Oliveira-Fernandes, R. M. Lavker, MicroRNAs of the mammalian eye display distinct and overlapping tissue specificity, Mol. Vis. 12, 1175-1184 (2006)). If the loss of outer segments was due to miRNA deficiency, re-expressing the relevant miRNA in the absence of DGCR8 should prevent the loss. The present inventors used next generation miRNA sequencing from isolated wild type cones at P60 to determine the most highly expressed miRNAs as candidates for controlling outer segment maintenance. We then designed a strategy to express miRNAs in the absence of DGCR8 in vivo. The expression pattern of miRNAs was highly uneven, with a single miRNA, miR-182 (S. Xu, P. D. Witmer, S. Lumayag, B. Kovacs, D. Valle, MicroRNA (miRNA) transcriptome of mouse retina and identification of a sensory organ-specific miRNA cluster, J. Biol. Chem. 282, 25053-25066 (2007); M. Karali, I. Peluso, V. Marigo, S. Banfi, Identification and characterization of microRNAs expressed in the mouse eye, Invest. Ophthalmol. Vis. Sci. 48, 509-515 (2007); N. J. Martinez, R. I. Gregory, Argonaute2 expression is post-transcriptionally coupled to microRNA abundance, RNA 19, 605-612 (2013)), representing 64% of all miRNA reads. Only four miRNAs were more abundant than 1%. Since miR-182 and miR-183 (third most abundant, 4% of reads) are processed, jointly with miR-96, from the same primary transcript and have related seed sequences (M. Karali, I. Peluso, V. Marigo, S. Banfi, Identification and characterization of microRNAs expressed in the mouse eye, Invest. Ophthalmol. Vis. Sci. 48, 509-515 (2007)), the inventors expressed these two together. The present inventors also expressed miR-124, since the lack of miR-124 has been shown to lead to cone death during development (R. Sanuki et al., miR-124a is required for hippocampal axogenesis and retinal cone survival through Lhx2 suppression, Nat. Neurosci. 14, 1125-1134 (2011)). To bypass the need for Drosha/DGCR8, the inventors generated short hairpin RNAs resembling pre-miRNAs (sh-miRs), which only need Dicer to produce mature miRNA mimics. They verified that expression of these sh-miRs in HEK293 cells leads to the specific repression of reporter mRNAs bearing miRNA sites. Next, using conditional AAVs, the inventors expressed either miR-182/183 or miR-124 mimics specifically in C-DGCR-KO cones at P30. MiR-182/183, but not miR-124, mimics prevented the loss of outer segments and cone opsins at P60. Therefore, the presence of miR-182/183 alone, in the absence of other miRNAs, is sufficient to maintain outer segments.

The inventors found that at P60 Argonaute protein level was strongly decreased in C-DGCR-KO cones. They also found that the expression of miR182/183 mimics following AAV administration at P60, hence after the loss of outer segments, did not restore outer segments at P90 in these mice. These results were expected since Argonaute protein level is often co-regulated with the cellular availability of miRNAs (M. Eiraku et al., Self-organizing optic-cup morphogenesis in three-dimensional culture, Nature 472, 51-56 (2011)), and without Argonautes the miRNA mimics would not exert their function. The present inventors therefore turned to an in vitro model system in which retinas were grown in 3D cultures from mouse embryonic stem cells (S. Siegert et al., Transcriptional code and disease map for adult retinal cell types, Nat. Neurosci. 15, 487-495, S1-2 (2012)) in order to investigate whether miR182/183 can induce the formation of outer segments. In these in vitro-built retinas, a well-separated photoreceptor layer, containing mostly rod-like cells, developed. However, no outer segments could be detected. Remarkably, the AAV-mediated expression of miR-183/96/182 cluster led to the appearance of outer segment-like membrane protrusions on the top of the photoreceptor layer. Similar to rod outer segments, rhodopsin, the sensory pigment of rods, was specifically localized into these protrusions.

To gain a mechanistic insight into the altered molecular pathways that lead to outer segment loss and decreased opsin expression, the present inventors followed the dynamics of changes in miRNA and mRNA expression in cones between P30 and P90. They isolated cones from C-DGCR-KO and wild type mice at five time points: P30, P40, P50, P60, and P90, and performed miRNA qPCR, next generation RNA sequencing (RNA-seq), and mRNA array experiments using RNA obtained from the isolated cones. They first confirmed that levels of miRNAs gradually decreased in C-DGCR-KO cones reaching 1-3% values at P90. As expected, the decrease of mature miRNAs was accompanied by accumulation of respective pri-miRNAs. Next the inventors followed mRNA expression in the same time window. Surprisingly, comparing gene expression at P30 and P60 in isolated C-DGCR-KO cones showed that 96.7% (RNA-seq) or 99.7% (mRNA arrays) of expressed genes changed less than two-fold; vast majority of genes involved in regulation of apoptosis belonged to this category. A small set of genes was, however, up or down regulated more than two-fold. Gene up regulation was expected since miRNAs are negative regulators of gene expression. Many of the up-regulated transcripts were predicted to be direct target of miR-182/183 and few other miRNAs expressed in cones.

The down regulation of genes was less expected. To determine if down-regulated genes were members of known pathways, the present inventors performed molecular pathway analysis of these transcripts. This analysis identified the genes in the phototransduction pathway as significantly down-regulated. In order to get a dynamic picture of the changes, they plotted the expression of the phototransduction pathway genes as a function of time in C-DGCR-KO and wild type mice from P30 to P90. In C-DGCR-KO cones, five phototransduction genes followed the same time course: unchanged or even up-regulated at P40 and decreasing gradually between P50 and P90. The inventors then asked if the loss of DGCR8 influences cone-specific genes more generally. A recent screen identified cone-specific genes in adult mice in an unbiased way (C. Punzo, K. Kornacker, C. L. Cepko, Stimulation of the insulin/mTOR pathway delays cone death in a mouse model of retinitis pigmentosa, Nat. Neurosci. 12, 44-52 (2009)), and they plotted the expression of these genes as a function of time in C-DGCR-KO and wild type mice. Remarkably, a large fraction, 34% (RNA-seq) or 38% (mRNA array), of cone-specific genes has been significantly down-regulated. The down regulation of cone-specific genes is highly significant, since any randomly selected similar number of genes showed no down regulation, but a slight up regulation. The decrease of expression followed two different time course patterns: most genes showed the same pattern as the five phototransduction genes, while a few genes decreased gradually from P30 to P90. The progression of the down regulation of most cone-specific genes was delayed compared to the start of the outer segment loss, making it likely that the decreasing expression of these genes is not the cause of outer segment loss. The down regulation is either caused by increased expression of an inhibitory factor which is regulated by miRNAs or directly by the Drosha/DGCR8 complex; or alternatively it is a secondary consequence of outer segment loss via a mechanism that regulates cone-specific gene expression depending on the length of cone outer segments.

In summary, the present inventors have identified some miRNA as necessary for the maintenance of cone outer segments, a subcellular compartment, and shown that miRNA depletion in adult cones leads to decreased expression of many cone-specific genes. The loss of cone outer segments is the final common pathway for many retinal diseases, the event that causes blindness. The findings that miR-183/96/182 cluster expression leads to the induction of outer segment-like structures in embryonic stem cell-derived retinal cultures, and that the same cluster is down regulated in several mouse models of the blinding disease retinitis pigmentosa (T. R. Sundermeier, K. Palczewski, The physiological impact of microRNA gene regulation in the retina, Cellular and Molecular Life Sciences 69, 2739-2750 (2012)), support that the re-expression of these miRNAs alone, or in combination with other factors (T. Léveillard, J.-A. Sahel, Rod-derived cone viability factor for treating blinding diseases: from clinic to redox signaling, Sci Transl Med 2, 26ps16 (2010); R. Wen et al., Regeneration of cone outer segments induced by CNTF, Adv. Exp. Med. Biol. 723, 93-99 (2012)) as a strategy to regenerate outer segments.

1-13. (canceled)

14. A method for treating a disease or condition associated with the downregulation of miRNA-182 (uuuggcaaugguagaacucacacu, SEQ ID NO: 1, or ugguucuagacuugccaacua, SEQ ID NO:2), miRNA-96 (uuuggcacuagcacauuuuugcu, SEQ ID NO:5, or aaucaugugcagugccaauaug, SEQ ID NO:6) and/or miRNA-183 (uauggcacugguagaauucacu, SEQ ID NO:3, or gugaauuaccgaagggccauaa, SEQ ID NO:4), such as ciliopathy or photoreceptor dysfunction, in a subject, the method comprising administering to the subject an effective amount of an agent that increases the expression of miRNA-182, miRNA-96 and/or miRNA-183 in the subject, wherein the agent is an isolated nucleic acid molecule comprising a nucleotide sequence coding for miRNA-182, miRNA-96 and/or miRNA-183.

15. The method of claim 14 further comprising the step of administering to the subject an additional factor such as Argonaute, Rod-derived Cone Viability Factor (RdCVF), and/or a nucleic acid molecule coding for such an additional factor.

16. The method of claim 14, wherein said ciliopathy is selected from the group comprising Senior-Løken syndrome, retinal degeneration, and retinitis pigmentosa.

17. The method of claim 14, wherein said photoreceptor dysfunction is selected from the group comprising achromatic vision, macular degeneration, retinitis pigmentosa, rod and/or cone dystrophy, and Usher syndrome.

18. The method of claim 14, wherein said miRNA is miRNA 182.

19. The method of claim 14, wherein said miRNA is miRNA 183.

20. The method of claim 14, wherein said miRNA is miRNA 96.

21. The method of claim 14, wherein the isolated nucleic acid molecule is administered to the subject in a form of a recombinant vector comprising the isolated nucleic acid molecule.

22. The method of claim 21, wherein the isolated nucleic acid molecule is administered to the subject in a form of a host cell comprising the recombinant vector.

23. The method of claim 14, wherein the isolated nucleic acid is administered to the subject in a form of a therapeutic composition.

24. The method of claim 23, wherein the composition further comprises a pharmaceutically acceptable carrier, diluent, or buffer.

25. The method of claim 24, wherein the carrier is a biodegradable polymer microsphere, a liposome, a colloidal gold particle, a lipopolysaccharide, a polypeptide, a polysaccharide, or a pegylated virus vehicle.

26. The composition according to claim 23, wherein the isolated nucleic acid is provided on a nanoparticle.


SEQUENCE LISTING (6 sequences)

SEQ ID NO: 1 - RNA, 24 nt, Homo sapiens
uuuggcaaugguagaacucacacu

SEQ ID NO: 2 - RNA, 21 nt, Homo sapiens
ugguucuagacuugccaacua

SEQ ID NO: 3 - RNA, 22 nt, Homo sapiens
uauggcacugguagaauucacu

SEQ ID NO: 4 - RNA, 22 nt, Homo sapiens
gugaauuaccgaagggccauaa

SEQ ID NO: 5 - RNA, 23 nt, Homo sapiens
uuuggcacuagcacauuuuugcu

SEQ ID NO: 6 - RNA, 22 nt, Homo sapiens
aaucaugugcagugccaauaug